SEQUENCE LISTING (22 sequences)

SEQ ID NO: 1 - RNA, 23 nt, synthetic construct
  source 1..23: mol_type = other RNA
  ncRNA 1..23: ncRNA_class = miRNA
aaaagtgcttacagtgcaggtag

SEQ ID NO: 2 - RNA, 23 nt, synthetic construct
  source 1..23: mol_type = other RNA
  ncRNA 1..23: ncRNA_class = miRNA
caaagtgcttacagtgcaggtag

SEQ ID NO: 3 - RNA, 23 nt, synthetic construct
  source 1..23: mol_type = other RNA
  ncRNA 1..23: ncRNA_class = miRNA
taaagtgcttatagtgcaggtag

SEQ ID NO: 4 - RNA, 23 nt, synthetic construct
  source 1..23: mol_type = other RNA
  ncRNA 1..23: ncRNA_class = miRNA
caaagtgctgttcgtgcaggtag

SEQ ID NO: 5 - protein, 8 aa, synthetic construct
HLSSQYSR

SEQ ID NO: 6 - protein, 12 aa, synthetic construct
APWHLSSQYSRT

SEQ ID NO: 7 - protein, 11 aa, synthetic construct
APWHLSSQYSR

SEQ ID NO: 8 - protein, 11 aa, synthetic construct
PWHLSSQYSRT

SEQ ID NO: 9 - protein, 10 aa, synthetic construct
PWHLSSQYSR

SEQ ID NO: 10 - protein, 12 aa, synthetic construct
APWHLSSQYSRT

SEQ ID NO: 11 - protein, 12 aa, synthetic construct
APYHLSSQYSRT

SEQ ID NO: 12 - protein, 11 aa, synthetic construct
PWHLSSQYSRT

SEQ ID NO: 13 - protein, 11 aa, synthetic construct
PYHLSSQYSRT

SEQ ID NO: 14 - protein, 10 aa, synthetic construct
WHLSSQYSRT

SEQ ID NO: 15 - protein, 10 aa, synthetic construct
YHLSSQYSRT

SEQ ID NO: 16 - protein, 9 aa, synthetic construct
WHLSSQYSR

SEQ ID NO: 17 - protein, 9 aa, synthetic construct
YHLSSQYSR

SEQ ID NO: 18 - protein, 8 aa, synthetic construct
HLSSQWSR

SEQ ID NO: 19 - protein, 12 aa, synthetic construct
APWHLSSQWSRT

SEQ ID NO: 20 - protein, 11 aa, synthetic construct
PWHLSSQWSRT

SEQ ID NO: 21 - protein, 10 aa, synthetic construct
WHLSSQWSRT

SEQ ID NO: 22 - protein, 9 aa, synthetic construct
WHLSSQWSR

(19) United States
(12) Patent Application Publication
(10) Pub. No.: US 2025/0346903 A1
(43) Pub. Date: Nov. 13, 2025

(54) USE OF CONJUGATES OF MICRORNA AND CARDIAC TARGETING PEPTIDES FOR TREATING HEART FAILURE

(71) Applicant: Georgetown University, Washington, DC (US)

(72) Inventor: Gary Ian Gallicano, Alexandria, VA (US)

(73) Assignee: Georgetown University, Washington, DC (US)

(21) Appl. No.: 18/870,532

(22) PCT Filed: May 31, 2023
(86) PCT No.: PCT/US2023/067644

§ 371 Date: Nov. 29, 2024

Related U.S. Application Data
(60) Provisional application No. 63/347,541, filed on May 31, 2022.

(51) Int. Cl. C12N 15/113; A61K 47/64; A61P 9/04

(52) U.S. Cl. CPC C12N 15/1137; A61K 47/64; A61P 9/04; C12N 2310/141; C12N 2310/3513; C12N 2320/32; C12Y 207/11017; C12Y 305/01098

(57) ABSTRACT

Methods of treating cardiac hypertrophy or cardiomyocyte hypertrophy, or methods of inhibiting progression of heart failure in a subject in need thereof, in which the methods comprise administering a pharmaceutical composition comprising an effective amount of a conjugate comprising microRNA and a cardiac targeting peptide. In addition, methods of inhibiting expression of Ca2+/calmodulin-dependent protein kinase II delta or histone deacetylase 4 in cardiomyocytes, in which the methods comprise contacting the cardiomyocytes with a conjugate comprising microRNA and a cardiac targeting peptide. The microRNA may be mi RN A 106a. miRNA17, miRNA20a, or miRNA93.

### CROSS-REFERENCE TO RELATED APPLICATIONS

This application claims the benefit of U.S. Provisional Application No. 63/347,541, filed on May 31, 2022, which is incorporated herein by reference in its entirety.

### SEQUENCE LISTING

The instant application contains a Sequence Listing which has been submitted electronically in XML format and is hereby incorporated by reference in its entirety. Said XML copy, created on May 26, 2023, is named “Georgetown041WO1,” and is 21,714 bytes in size.

### BACKGROUND OF THE INVENTION

Etiologies of heart failure have been investigated for over a century culminating in data that have led to numerous pharmacological and surgical therapies. Unfortunately, to date, even with the most current treatments, progressive heart failure is still associated with an annual mortality rate in the U.S. of 600,000 people, and those living with the disease have only a marginal improvement in quality of life.

Recent technological advances have brought about new paradigms for treating many diseases, like heart failure, that previously had been extremely difficult to resolve. One of these new paradigms has been a shift from pharmacological agents to antisense technology (e.g., microRNA (miRNA)) that molecularly suppress disease onset. Although this paradigm shift may have been postulated over a decade ago (van Rooij et al., 2008), only within the past few years has it become feasible. Now, antisense technologies have shown promise for treating Huntington's disease and muscular dystrophy (Pfister et al., 2017), cancers (Rupaimoole & Slack, 2017), and some viral infections (Matsui & Corey, 2017; Adams et al., 2017).

While miRNAs have demonstrated promise as a therapy, development for its use for clinical applications in heart failure is challenging. Because the overall mechanisms involved in the disease is complex, with multiple pathways that are not fully understood, identifying the mis-expressed genes in heart failure to target is difficult. And even if target genes can be determined, it can also be challenging to identify which miRNA can effectively alter the expression of the targeted gene. Further, it is critical to determine how the miRNA can be delivered to the heart. An miRNA therapy that can meet all of these challenges remains as an unmet need.

### SUMMARY OF THE INVENTION

The present application is directed to methods and compositions for heart failure.

In one aspect, the present invention relates to a method of treating cardiac hypertrophy in a subject in need thereof, in which the method comprises administering a pharmaceutical composition comprising an effective amount of a conjugate comprising microRNA and a cardiac targeting peptide (CTP). In another aspect, the present invention relates to a pharmaceutical composition comprising an effective amount of a conjugate comprising microRNA and a CTP, for use in treating cardiac hypertrophy in a subject in need thereof. In some embodiments, the cardiac hypertrophy is induced by angiotensin or phenylephrine.

In another aspect, the present invention relates to a method of treating cardiomyocyte hypertrophy in a subject in need thereof, in which the method comprises administering a pharmaceutical composition comprising an effective amount of a conjugate comprising microRNA and a cardiac targeting peptide (CTP). In yet another aspect, the present invention relates to a pharmaceutical composition comprising an effective amount of a conjugate comprising microRNA and a CTP, for use in treating cardiomyocyte hypertrophy in a subject in need thereof. In some embodiments, the cardiomyocyte hypertrophy is induced by angiotensin or phenylephrine.

In a further aspect, the present invention relates to a method of inhibiting progression of heart failure in a subject in need thereof, in which the method comprises administering a pharmaceutical composition comprising an effective amount of a conjugate comprising microRNA and a CTP. In yet a further aspect, the present invention relates to a pharmaceutical composition comprising an effective amount of a conjugate comprising microRNA and a CTP, for use in inhibiting progression of heart failure in a subject in need thereof.

In a further aspect, the present invention relates to a method of reversing a reduction in cardiac function in a subject in need thereof, in which the method comprises administering a pharmaceutical composition comprising an effective amount of a conjugate comprising microRNA and a CTP. In another aspect, the present invention relates to a pharmaceutical composition comprising an effective amount of a conjugate comprising microRNA and a CTP, for use in reversing a reduction in cardiac function in a subject in need thereof.

In another aspect, the present invention is directed to a method of preventing a further reduction in cardiac function in a subject in need thereof, the method comprising administering a pharmaceutical composition comprising an effective amount of the conjugate comprising a nucleic acid molecule and a cardiac targeting peptide. In an aspect, the present invention relates to a pharmaceutical composition comprising an effective amount of a conjugate comprising microRNA and a CTP, for use in preventing a further reduction in cardiac function in a subject in need thereof.

In some embodiments, the subject is already determined to have a reduction in cardiac function prior to administration of the pharmaceutical composition. In certain embodiments, cardiac function is measured by ejection fraction (EF), fractional shortening (FS), left ventricular mass (LVmass), or a combination thereof.

In an additional aspect, the present invention relates to a method of inhibiting expression of Ca²⁺/calmodulin-dependent protein kinase II delta (CaMKIIδ) in cardiomyocytes, or a method of inhibiting expression of histone deacetylase 4 (HDAC4) in cardiomyocytes, in which the method comprises contacting the cardiomyocytes with a conjugate comprising microRNA and a CTP. In another aspect, the present invention relates to a conjugate comprising a microRNA and a CTP, for use in inhibiting expression of CaMKIIδ, or for use in inhibiting HDAC4, in cardiomyocytes.

The microRNA may be selected from miRNA106a, miRNA17, miRNA20a, and miRNA93. In some embodiments, the microRNA is miRNA106a.

In some embodiments, the CTP comprises an amino acid sequence of HLSSQYSR (SEQ ID NO: 5) or HLSSQWSR (SEQ ID NO: 18). In certain embodiments, the CTP has an amino acid sequence of APWHLSSQYSRT (SEQ ID NO:6).

In some embodiments, the nucleic acid molecule and the CTP are linked by a covalent bond or a non-covalent bond. In certain embodiments, the nucleic acid molecule and the CTP are linked by a linker molecule. The linker molecule may comprise a cleavage site.

### BRIEF DESCRIPTION OF THE DRAWINGS

FIG. 1 shows effects of microRNA-17 (miRNA17), miRNA20a, miRNA93, and miRNA106a on human cardiomyocytes (HCMs) treated with phenylephrine (PE) and angiotensin 2 (Ang2 or AngII) (the combination referred to herein as “PE/Ang2” or “PE/AngII” or “Ang2/PE” or “AngII/PE”), as described in Example 1. Panels A-D are microscopic images of untreated HCMs (Panel A); HCMs treated with PE/Ang2 for 144 hours (Panel B); HCMs treated with PE/Ang2 for 144 hours, and then transfected with miRNA control (Panel C); and HCMs treated with PE/Ang2 for 144 hours, and then treated with miRNA106a. The insets in Panels A, B, and D show anti-desmin (cardiac) staining in same cells; each stained shape represents how the area of the cells was measured. Panel E provides measurements of cell size for PE/Ang2-treated HCMs, and PE/Ang2-treated HCMs that were also treated with the miRNAs. Panels F-K show Western blots (Panels F, H, and J) and quantified protein expression levels (Panels G, I, and K) of CamKIIδ (Panel F and G), histone deacetylase 4 (HDAc4) (Panel H and I), and cardiac actin (Panel J and K) in PE/Ang2-treated HCMs, and PE/Ang2-treated HCMs that were also treated with the miRNAs (n=6 for each experiment).

FIG. 2 shows assay results of CamKIIδ (Panels A and D), HDAC4 (Panels B and E), and G protein-coupled receptor kinase-2 (GRK2) (Panels C and F) 3′ untranslated regions-(3′UTR)-luciferase constructs that were transfected by miRNA106a using Transit2 reagent (Panels A-C) or by a conjugate comprising miRNA106a and a CTP having the amino acid sequence of APWHLSSQYSRT (SEQ ID NO:6) (the “CTP-miRNA106a conjugate”) (Panels D-F), as described in Example 2 (n=6 for each experiment).

FIG. 3 shows the results of introducing a Cy5.5-CTP-miRNA106a conjugate to HCMs and human embryonic kidney 293 (HEK293) cells, as described in Example 2. Panels A-D are images of detected immunofluorescence for Cy5.5 (left images), desmin (middle images), and 4′,6-diamidino-2-phenylindole (DAPI) (right images) for HCMs treated with the CTP-miRNA106a conjugate (Panel A), control HCMs (Panel B), HCMs treated with the CTP (Panel C), and HEK293 cells treated with the CTP (Panel D). Panel E shows real-time reverse transcription polymerase chain reaction (RT-PCR) results of HCMs and HEK293 cells untreated, treated with 2 nM or 200 nM of miRNA106a using TransIT-X2 transfection reagent, or treated with 0.5 μg/ml or 5 μg/ml of the CTP-miRNA106a conjugate. Panel F shows results of fluorescence-activated cell sorting (FACS) analysis of HCMs treated with the 5 μg of the CTP-miRNA106a conjugate (square-marked line) or 5 μM of the CTP alone (circle-marked line), or untreated (plain line). Panel G shows results of FACS analysis of HCMs treated with 5 μg of the CTP-miRNA106a conjugate (square-marked line) or 5 μM of the CTP alone (circle-marked line), or untreated (star-marked line); and HEK293 cells treated with 5 μg of the CTP-miRNA106a conjugate (triangle-marked line) or 5 μM of the CTP alone (plain line), or untreated (diamond-marked line). Panel H shows results of FACS analysis of treatment of 5 μg of the CTP-miRNA conjugate in HCMs (square-marked line), endothelial cells (ECs) (star-marked line), and human cardiac fibroblasts (HCFs) (triangle-marked line); and untransfected HCMs (circle-marked line), ECs (plain line), and HCFs (diamond-marked line). (n=9 for RT-PCR; n=3 for FACS analysis).

FIG. 4 shows effects on expression of various proteins in HCMs after treatment with miRNA106a or the CTP-miRNA106a conjugate, as described in Example 2. Panels A and B show Western blots (Panel A) and quantified protein expression levels (Panel B) of CamKIIδ, HDAC4, brain natriuretic peptide (BNP), and cardiac troponin (cTnnT) in HCMs treated with PE/Ang2 (24, 72, and 144 hours), and in HCMs treated with PE/Ang2 for 144 hours and treated with the CTP-miRNA106a conjugate (six runs; p<0.05)). Panels C-F are images of detected immunofluorescence for anti-HDAC4 in untreated HCMs (Panel C), HCMs treated with PE/Ang2 for 72 hours (Panel D), HCMs treated with PE/Ang2 for 72 hours and miRNA106a for 24 hours (Panel E), and HCMs treated with PE/Ang2 for 72 hours and the CTP-miRNA106a conjugate for 24 hours (Panel F). Arrows point to the nucleus of HCMs in Panels C and F, and to the cytoplasm of HCMs in Panels D and E.

FIG. 5 shows the effects of the miRNAs on GRK2 signaling in HCMs treated with PE/Ang2, as described in Example 3. Panels A and B show Western blots (Panel A) and quantified protein expression levels (Panel B) of GRK2 in HCMs treated with PE/Ang2 for 72 hours and treated with miRNA17, miRNA20a, miRNA93, and miRNA106a. Panels C and D show Western blots (Panel C) and quantified protein expression levels (Panel D) of GRK2 in HCMs treated with PE/Ang2 for 72 hours and treated with the CTP-miRNA106a conjugate for 24 hours. Panel E shows Western blot showing ubiquitinated GRK2 identified using anti-ubiquitin (rabbit). The experiment was run three times.

FIG. 6 shows the effect of CTP-conjugated microRNAs on mitochondrial health, as described in Example 4. Panels A-E are images and quantification of HCMs stained with JC-1 dye to identify health and unhealthy. The HCMs were untreated (Panel A), treated with 10 nM Ang2 and 200 μM PE for 72 hours (Panel B), treated with ten times the amount of Ang2/PE normally used to induce hypertrophy (Panel C), transfected with 200 nM of miRNA106a and cultured for 72 hours (Panel D), and treated with 5 μg/ml of the CTP-miRNA106a conjugate for 72 hours (Panel E) (scale bars=100 μm for all panels).

FIG. 7 shows Western blots analyses to identify mitofusin 2 (Mfn2) protein expression HCMs treated with the CTP-miRNA106a conjugate, as described in Example 4. Panels A and B show Western blots (Panel A) and quantitative analysis (Panel B) of HCMs cultured in three increasing dosages (0.5 μg/ml, 5 μg/ml, 50 μg/ml) of the CTP-miRNA106a conjugate for 72 hours. Panels C and D show Western blots (Panel C) and quantitative analysis (Panel D) of HCMs cultured in three increasing transfection concentrations (20 nM, 200 nM, 2 μM) of the CTP-miRNA106a conjugate for 72 hours (n=6 for each Western blot).

FIG. 8 shows the effect of the CTP-miRNA106a conjugate on nuclear factor kappa-B (NfκB) translocation into the nucleus of HCMs treated with PE/Ang2, as described in Example 5. Panels A, C, and E are images of untreated HCMs (Panel A), HCMs treated with PE/Ang2 for three hours (Panel C), and HCMs pretreated with the CTP-miRNA106a conjugate followed by treatment with PE/Ang2 for 3 hours (Panel E), in which the left images display NfκB staining, the middle images display DAPI staining, and the right images display merged (DAPI/anti-NfκB) staining. Panel B, Panel D, and Panel F are high magnification views of the right images of Panel A, Panel C, and Panel E, respectively. Panel G shows results of analyzing all cells within the images of Panels B, D, and F, using ImageJ to measure red pixel intensity on a 0 (black) to 255 (highest red intensity) scale, and then calculating the ratio of four 11×11 pixel² regions/nucleus to four regions/cytoplasm. Panel H and Panel I show Western blots of NfkB (anti-p65) and inhibitory subunit of NFκB-α (Iκα), respectively.

FIG. 9 shows the effect of the CTP-miRNA106a conjugate on Ang2/PE-induced NfκB gene activity, as described in Example 5. Panel A shows a comparison of luciferase expression in untreated HCMs, HCMs treated with Ang2/PE for three hours, and HCMs treated with Ang2/PE for three hours and pretreated with the CTP-miRNA106a conjugate for 24 hours. Panel B shows a comparison of luciferase expression in untreated HCMs, HCMs treated with tumor necrosis factor-α (TNF-α) for three hours, and HCMs treated with TNF-α for three hours and pretreated with the CTP-miRNA106a conjugate for 24 hours. Panel C shows a comparison of luciferase expression in untreated HEK293 cells, HEK293 cells treated with TNF-α for three hours, and HEK293 cells treated with TNF-α for three hours and pretreated with the CTP-miRNA106a conjugate for 24 hours. Panel D shows a comparison of luciferase expression in untreated HCMs, HCMs treated with Ang2/PE for 24 hours, and HCMs treated with Ang2/PE for 24 hours and transfected with miRNA17, miRNA20a, miRNA93, or miRNA106a.

FIG. 10 shows the effect of the CTP-miRNA106a conjugate on genes activated by NfκB in Ang2/PE-treated HCMs, as described in Example 5. Panels A-D show analysis by FACS of interleukin-1 (IL-1β) production in HCMs that are untreated; treated with Ang2/PE for 24 hours; treated with the CTP-miRNA106a conjugate for 24 hours followed by treatment with Ang2/PE for 24 hours; treated with Ang2/PE for 48 hours with treatment with the CTP-miRNA106a conjugate beginning after 24 hours; and transfected with miRNA106a and then treated with Ang2/PE for 24 hours. Panels E-H show analysis by FACS of interleukin-6 (IL-6) production in HCMs that are untreated; treated with Ang2/PE for 24 hours; treated with the CTP-miRNA106a conjugate for 24 hours followed by treatment with Ang2/PE for 24 hours; treated with Ang2/PE for 48 hours with treatment with the CTP-miRNA106a conjugate beginning after 24 hours; and transfected with miRNA106a and then treated with Ang2/PE for 24 hours. Panels I-L show analysis by FACS of TNF-α production in HCMs that are untreated; treated with Ang2/PE for 24 hours; treated with the CTP-miRNA106a conjugate for 24 hours followed by treatment with Ang2/PE for 24 hours; treated with Ang2/PE for 48 hours with treatment with the CTP-miRNA106a conjugate beginning after 24 hours; and transfected with miRNA106a and then treated with Ang2/PE for 24 hours.

FIG. 11 shows effects of the CTP-microRNA106a conjugate on Ang2/PE-induced phospholipase C beta 1 (PLCβ1) expression, as described in Example 6. Panel A shows a Western blot displaying PLCβ1 expression in HCMs treated with Ang2/PE for 0 hours (untreated), 24 hours, 72 hours, and 144 hours, and in HCMs treated with Ang2/PE for 144 hours and with the CTP-microRNA106a conjugate for 72 hours; anti-desmin was used as a loading control. Panel B shows results of analyzing the pixel intensity of each band of the Western blot in Panel A using ImageJ (n=3 for 0-to-144-hour treatment with Ang2/PE, n=8 for 144-hour treatment with Ang2/PE and 72-hour treatment with the CTP-microRNA106a conjugate). Panel C shows results of transfecting HEK293 cells with a plasmid containing a CMV promoter driving luciferase linked to the 3′UTR of PLCβ1, and further transfection 24 hours later with miRNA106a or miRNA93, or with no further transfection (control). Panel D shows Western blot displaying PLCβ1 expression in HCMs treated with Ang2/PE for 0 hours (untreated), 24 hours, or 72 hours, or treated with four siRNAs verified to target PLCβ1.

FIG. 12 shows protein kinase C (PKC) localization in HCMs treated with Ang2/PE and the CTP-microRNA106 conjugate, as described in Example 6. Panels A, B, C, and D are images of untreated HCMs (Panel A); HCMs treated with phorbal myristate acetate (PMA), a synthetic activator of PKC, for 30 minutes (Panel B); HCMs treated with Ang2/PE for 24 hours (Panel C); and HCMs treated with Ang2/PE for 24 hours and treated with the CTP-miRNA106a conjugate for 48 hours (Panel D). For each panel, the left image displays anti-PKCα staining, with arrows pointing to cells exhibiting membrane localization of PKC; the middle image shows anti-desmin staining, which marks the intermediate filament specific to cardiomyocytes; and the right image of anti-PKCα, anti-desmin, and DAPI staining.

FIG. 13 shows PKC activity determined using a PKC kinase activity kit (ADI-EKS-420A) from Enzo Life Science Inc. in HCMs that are untreated, treated with Ang2/PE, and treated with Ang2/PE and the CTP-microRNA106a conjugate for one hour (Panel A), three hours (Panel B), 24 hours (Panel C), and 72 hours (Panel D), as described in Example 6. The results are presented as normalized PKC activity ratios to untreated HCMs. Panel E presents a smooth curved graph of PKC activity over time for the untreated HCMs, HCMs treated with Ang2/PE, and HCMs treated with Ang2/PE and the CTP-microRNA106a conjugate.

FIG. 14 shows presence of gap junction protein Connexin 43 (CNX43) and CNX43 phosphorylation in untreated HCMs (Panels A and B), HCMs treated with 100 mM PMA (Panels C and D), HCMs treated with Ang2/PE for 24 hours (Panels E and F), and HCMs treated with Ang2/PE for 72 hours and the CTP-microRNA106 conjugate for 48 hours (Panels G and H), as described in Example 6. Panels A, C, E, and G show HCMs stained with anti-CNX43, with arrows pointing to confluent HCMs. Panels B, D, F, and H show HCMs stained with an antibody directed specifically to the PKC phosphorylated serine (Ser368), with arrows pointing to phospho-CNX43 positive gap junctions.

FIG. 15 shows measurements of ejection fraction (EF) (Panels A, D, and G), fractional shortening (FS) (Panels B, E, and H), and changes in left ventricle mass (LVmass) (Panels C, F, and I) in mice experiencing heart failure induced by Ang2 and isoproterenol (Iso) (the combination referred to herein as “Ang2/Iso”) and administered the CTP-microRNA106a conjugate. Panels A-C show results for a first cohort comprising mice that were delivered saline (n=2), 500 μg/kg/d Ang2 and 30 ng/kg/d Iso (n=2), and 500 μg/kg/d Ang2 and 30 ng/kg/d Iso along with injections of 10 mg/kg of the CTP-miRNA106a conjugate at weeks 5, 6, 7, and 8 post-Ang2/Iso delivery (n=2). Panels D-F show result for a second cohort comprising mice that were delivered saline (n=3), 2 mg/kg/d Ang2 and 30 ng/kg/d Iso (n=4), and 2 mg/kg/d Ang2 and 30 ng/kg/d Iso along with injections of 10 mg/kg of the CTP-miRNA106a conjugate at weeks 3 and 4 post-Ang2/Iso delivery (n=2). Panels G-I show results for a third cohort comprising mice that were delivered saline (n=1), received no delivery of saline or Ang2/Iso (n=2); 1.5 mg/kg/d Ang2 and 30 ng/kg/d Iso (n=2), and 1.5 mg/kg/d Ang2 and 30 ng/kg/d Iso along with injections of 10 mg/kg of the CTP-miRNA106a conjugate at weeks 2, 3, 4, 5, 6, and 7 post-Ang2/Iso delivery (n=2).

### DETAILED DESCRIPTION OF THE INVENTION

The practice of the present invention will employ, unless otherwise indicated, conventional techniques of pharmaceutics, molecular biology, cell biology, protein chemistry, and biotechnology, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

The terms “inhibit,” “reduce,” and “decrease” are used interchangeably and refer to any statistically significant decrease in occurrence or activity, including full blocking of the occurrence or activity. For example, “inhibition” can refer to a decrease of about 10%, 20%, 30%, 40%, 50%, 60%, 70%, 80%, 90% or 100% in activity or occurrence. An “inhibitor” is a molecule, factor, or substance that produces a statistically significant decrease in the occurrence or activity of a process, pathway, or molecule.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. In certain embodiments, a subject is successfully “treated” for a disease or disorder if the patient shows total, partial, or transient alleviation or elimination of at least one symptom or measurable physical parameter associated with the disease or disorder.

“Prevent” or “prevention” refers to prophylactic or preventative measures that prevent and/or slow the development of a targeted pathologic condition or disorder. Thus, those in need of prevention include those at risk of or susceptible to developing the disorder.

An “effective amount” of an active agent is an amount sufficient to carry out a specifically stated purpose.

An “active agent” is an ingredient that is intended to furnish biological activity. The active agent can be in association with one or more other ingredients.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g., acetate, phosphate, or citrate buffer), a surfactant (e.g., polysorbate), a stabilizing agent (e.g., polyol or amino acid), a preservative (e.g., sodium benzoate), and/or other conventional solubilizing or dispersing agents.

“Nucleic acid molecule” refers to an oligonucleotide chain comprising individual nucleic acid residues (e.g., nucleotides and/or nucleosides). The nucleic acid residues may consist or comprise RNA, or may consist or comprise DNA.

As used herein, “microRNA” or “miRNA” refers to a small single-stranded non-coding RNA molecule, and usually comprises about 15 to 25 nucleotides. Typically, microRNA targets an mRNA using a “seed” sequence of seven to eight bases that are complementary to both the miRNA and the mRNA. This targeting usually occurs within the 3′ UTR of the mRNA.

As used herein, “small interfering RNA” or “siRNA” refers to single-stranded or double-stranded RNA that is non-coding, and usually comprises about 15 to 25 base pairs, or in some embodiments about 20 to 24 base pairs, in length. Often, small interfering RNA are designed as an exact complementary sequence on the mRNA it targets.

“Aptamer” refers to an oligonucleotide that binds to a specific target molecule. The aptamer is typically generated through an in vitro selection methods such as SELEX (systematic evolution of ligands by exponential enrichment).

“Cardiac targeting peptide” or “CTP” refers to a peptide that is able to transfect cardiomyocytes without the use of a transfection reagent (for example, a lipid-based transfection reagent such as lipofectamine 3000).

“Cardiac hypertrophy” refers to the thickening of the ventricular myocardium due to physiological or pathophysiological events. The cardiac muscle fibers thicken and/or cells become enlarged, causing an increase in cardiac muscle mass.

“Cardiomyocyte hypertrophy” refers to the enlargement of the volume of a cardiomyocyte, which often occurs to compensate for a physiological decrease in cell function.

“Heart failure” refers to a condition that develops when the heart does not pump enough blood for the body's needs. Heart failure can occur if the heart cannot fill up with enough blood, or if the heart is too weak to pump properly.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

### Methods of the Invention

The present invention is directed to uses of a conjugate comprising a nucleic acid molecule and a CTP. As shown in the Examples, the conjugate can target and inhibit expression of proteins involved in heart failure such as CaMKIIδ and HDAC4, and well as unexpectedly reverse the hypertrophic response to PE and Ang2 in HCMs. These results demonstrate that the conjugate can be used to treat cardiac hypertrophy, treat cardiomyocyte hypertrophy, or inhibit progression of heart failure, as well as inhibit expression of proteins involved in heart failure.

Thus, in one aspect, the present invention is directed to a method of treating cardiac hypertrophy in a subject in need thereof. In another aspect, the present invention is directed to a method of treating cardiomyocyte hypertrophy in a subject in need thereof. These methods comprise administering a pharmaceutical composition comprising an effective amount of the conjugate comprising a nucleic acid molecule and a cardiac targeting peptide.

The cardiac hypertrophy or the cardiomyocyte hypertrophy may occur from physiological hypertrophy (e.g., resulting from exercise or pregnancy) or from pathological hypertrophy. In some embodiments, the cardiac hypertrophy or the cardiomyocyte hypertrophy may be pathological hypertrophy caused by, for example, hypertension or valvular disease.

In yet another aspect, the present invention is directed to a method of inhibiting progression of heart failure in a subject in need thereof, the method comprising administering a pharmaceutical composition comprising an effective amount of the conjugate comprising a nucleic acid molecule and a cardiac targeting peptide.

In some embodiments, the inhibition of progression of heart failure may be demonstrated by prevention of one or more symptoms of heart failure from worsening, for example, from increasing in magnitude, frequency, or duration. Symptoms of heart failure include, but are not limited to, dyspnea (shortness of breath), coughing or wheezing, elevated high rate, edema (build-up of fluid), nausea or lack of appetite, fatigue or feeling light-headed, confusion or impaired thinking, and ant combination thereof.

In some embodiments, the inhibition of progression of heart failure may be demonstrated by preventing the severity of heart failure from increasing according to the New York Heart Association (NYHA) classification system, which is reproduced in Table 1. In certain embodiments, inhibition of progression of heart failure is demonstrated by preventing the subject's symptoms from increasing to a higher class under the NYHA classification system. In other embodiments, inhibition of progression of heart failure is demonstrated by preventing the subject's symptoms from increasing to Class III, or increasing to Class IV, under the NYHA classification system.

TABLE 1
NYHA classification of heart failure.

| Class | Symptoms |
|---|---|
| Class I | No limitation of physical activity |
| (mild) | Ordinary physical activity does not cause undue fatigue, |
| | palpitation (rapid/irregular heartbeat), or dyspnea. |
| Class II | Slight limitation of physical activity |
| (mild) | Comfortable at rest, but ordinary physical activity results in |
| | fatigue, palpitation, or dyspnea. |
| Class III | Marked limitation of physical activity |
| (moderate) | Comfortable at rest, but less than ordinary physical activity |
| | causes fatigue, palpitation, or dyspnea. |
| Class IV | Unable to carry out any physical activity without discomfort |
| (severe) | Symptoms of fatigue, palpitation, or dyspnea are present at |
| | rest |
| | If any physical activity is undertaken, discomfort increases |

In a further aspect, the present invention is directed to a method of reversing a reduction in cardiac function in a subject in need thereof. In yet another aspect, the present invention is directed to a method of preventing a further reduction in cardiac function in a subject in need thereof. These methods comprising administering a pharmaceutical composition comprising an effective amount of the conjugate comprising a nucleic acid molecule and a cardiac targeting peptide. In some embodiments, the subject is already determined to have a reduction in cardiac function prior to administration of the pharmaceutical composition.

The reduction in cardiac function may be characterized by or due to a change in a measurement associated with cardiac function, for example, EF, FS, LVmass, or a combination thereof. Such a change may be a decrease or increase to levels known in the art as being not normal or outside a normal range, which may take into consideration such factors as the subject's weight, age, gender, etc. In some embodiments, the reduction in cardiac function may be characterized by or due to a decrease in EF, such as to an EF below about 50% or about 55%. In some embodiments, the reduction in cardiac function may be characterized by or due to a decrease in FS, such as to an FS below about 25% or about 30%. In some embodiments, the reduction in cardiac function may be characterized by or due to an increase in LVmass, such as to an LVmass above about 205 g or about 210 g or about 215 g for men, and about 155 g or about 160 g or about 165 g for women; or, normalized to body surface area, about 105 g/m² or about 110 g/m² or about 115 g/m² for men, and about 95 g/m² or about 100 g/m² or about 105 g/m² for women.

In some embodiments, reversing the reduction in cardiac function may comprise or result in, for example, an increase in EF and/or FS, or an increase in EF and/or FS to within the normal range; and/or a decrease in LVmass, or a decrease in LVmass to within the normal range.

In some embodiments, preventing a further reduction in cardiac function may comprise or result in, for example, inhibiting a further decrease in EF and/or FS, and/or inhibiting a further increase in LVmass.

The efficacy of a pharmaceutical composition or method of the invention can be demonstrated or assessed using standard methods known in the art, such as methods that compare the efficacy of a given/“test” composition or method to a “control” composition or method. For example, the efficacy of a given composition or method in treating cardiac hypertrophy may be demonstrated or assessed by comparing its ability to improve one or more clinical indicators or symptoms of cardiac hypertrophy as compared to that of a control composition or control method, such as a placebo control. For instance, a comparison can be made between different subjects (e.g., between a test group of subjects or a control group of subjects). Similarly, the efficacy of a given composition or method in treatment can be demonstrated or assessed in a single subject by comparing one or more clinical indicators or symptoms of cardiac hypertrophy in the subject before and after treatment.

In some embodiments, the subject is a human, a non-human primate, a mouse, a rat, a dog or a cat. In preferred embodiments, the subject is a human.

In carrying out the methods described herein, any suitable method or route of administration can be used to deliver the active agents or combinations thereof described herein. The term “administration” includes any route of introducing or delivering the specified compositions or agents to subjects.

In some embodiments, the conjugate, or pharmaceutical compositions thereof, may be administered by any route, for example, by infusion or injection, orally, by absorption through epithelial or mucocutaneous linings (e.g., oral mucosa, rectal and intestinal mucosa, etc.), etc. Administration can be systemic or local. Various delivery systems are known, e.g., encapsulation in liposomes, microparticles, microcapsules, capsules, etc., and can be used to administer the conjugate.

In certain embodiments, the conjugate is administered parenterally. Parenteral routes of administration include intravenous (IV), intramuscular, intraperitoneal, intrathecal, and subcutaneous.

In embodiments of the invention, the pharmaceutical composition can be administered in an “effective amount.” The amount of the conjugate that will be effective in the methods described herein will depend on the nature or extent of the subject, and can be determined by standard clinical techniques. In addition, in vitro or in vivo assays may be employed to identify optimal dosage ranges. The precise dose to be employed in the formulations of the present invention will also depend on the route of administration and the extent of the condition, and dosing should be decided according to the judgment of the practitioner and each patient's circumstances. One of skill in the art can readily perform dosing studies (whether using single agents or combinations of agents) to determine appropriate doses to use, for example using assays such as those described in the Examples section of this patent application, such as animal subjects routinely used in the pharmaceutical sciences for performing dosing studies.

For example, in some embodiments the dose of the conjugate may be calculated based on studies in humans or other mammals carried out to determine efficacy and/or effective amounts of the conjugate. The dose amount and frequency or timing of administration may be determined by methods known in the art and may depend on factors such as the pharmaceutical form, route of administration, whether it is used in combination with other active agents (for example, the dosage of the conjugate required may be lower when it is used in combination with another active agent), and patient characteristics including age, body weight, or the presence of any medical conditions affecting drug metabolism. In those embodiments described herein that refer to specific doses of the conjugate to be administered based on mouse studies, one of skill in the art can readily determine comparable doses for human studies based on the mouse doses, for example using the types of dosing studies and calculations described herein. In some embodiments suitable doses of the conjugate described herein can be determined by performing dosing studies of the type that are standard in the art, such as dose escalation studies, for example using the dosages shown to be effective in mice in the Examples section of this patent application as a starting point.

Dosing regimens can also be adjusted and optimized by performing studies of the type that are standard in the art, for example using the dosing regimens shown to be effective in mice in the Examples section of this patent application as a starting point. In some embodiments the active agents are administered daily, or twice per week, or weekly, or every two weeks, or monthly.

In yet another aspect, the present invention is directed to methods of inhibiting expression of one or more proteins involved in heart failure in a cardiomyocyte. The methods comprise contacting the cardiomyocyte with the conjugate.

In some embodiments, the one or more proteins involved in heart failure may be selected from CaMKIIδ, HDAC4, GRK2, protein kinase A (PKA), signal transducer and activator of transcription 3 (STAT3), friend of Gata 2 (FOG2), phospholipase C-beta (PLC-β), IL-1β, IL-6, TNF-α, and any combination thereof. In certain embodiments, the one or more proteins involved in heart failure may be selected from CaMKIIδ, HDAC4, GRK2, and PLC-β, and any combination thereof. In preferred embodiments, the one or more protein involved in heart failure may be selected from CaMKIIδ and/or HDAC4.

In a further aspect, the present invention is directed to methods of inhibiting or preventing translocation of NfκB to the nucleus in cardiomyocytes, or methods of inhibiting or preventing NfκB activity in the nucleus of cardiomyocytes. The methods comprise contacting the cardiomyocyte with the conjugate.

In another aspect, the present invention is directed to methods of inhibiting or preventing expression of PLCβ1 in cardiomyocytes, or methods of inhibiting or preventing translocation of PKC to the plasma membrane in cardiomyocytes, or methods of inhibiting or preventing PKC activity in cardiomyocytes. The methods comprise contacting the cardiomyocyte with the conjugate.

In embodiments of the invention, the cardiomyocytes are hypertrophic.

### Nucleic Acid-CTP Conjugates

The conjugates of the present invention comprise a nucleic acid molecule and a CTP.

In some embodiments, the nucleic acid molecule targets one or more proteins that are involved in heart failure, including, but not limited to, CaMKIIδ, HDAC4, GRK2, PKA, STAT3, FOG2, and PLC-β. In certain embodiments, the nucleic acid molecule targets CaMKIIδ, HDAC4, GRK2, or PLC-β. In preferred embodiments, the nucleic acid molecule targets CaMKIIδ and/or HDAC4.

In some embodiments, the nucleic acid molecule may be miRNA, siRNA, or a DNA or RNA aptamer. In preferred embodiments, the nucleic acid molecule is miRNA.

In some embodiments, the miRNA may be selected from miRNA106a, miRNA17, miRNA20a, and miRNA93. The nucleotide sequences for these miRNAs are provided in Table 2 below.

TABLE 2
MiRNA sequences.

| miRNA | RNA Sequence | SEQ ID NO |
|---|---|---|
| miRNA106a | AAAAGUGCUUACAGUGCAGGUAG | 1 |
| miRNA17 | CAAAGUGCUUACAGUGCAGGUAG | 2 |
| miRNA20a | UAAAGUGCUUAUAGUGCAGGUAG | 3 |
| miRNA93 | CAAAGUGCUGUUCGUGCAGGUAG | 4 |

The nucleic acid molecule is conjugated to a CTP. In some embodiments, the CTP comprises the amino acid sequence HLSSQYSR (SEQ ID NO: 5). In certain embodiments, the CTP comprising the amino acid sequence HLSSQYSR (SEQ ID NO: 5) is about 8 to 12 amino acids in length, or about 8 to 10 amino acids in length; examples of such CTPs include, but are not limited to, CTPs having the amino acid sequences listed in Table 3. In certain embodiments, the CTP consists of the amino acid sequence HLSSQYSR (SEQ ID NO: 5).

TABLE 3
Sequences of CTPs comprising HLSSQYSR (SEQ ID
NO: 5), and that are conjugated to the nucleic
acid molecule.

| CTP SEQUENCE | SEQ ID NO |
|---|---|
| APWHLSSQYSRT | 6 |
| APWHLSSQYSR | 7 |
| PWHLSSQYSRT | 8 |
| PWHLSSQYSR | 9 |
| APWHLSSQYSRT | 10 |
| APYHLSSQYSRT | 11 |
| PWHLSSQYSRT | 12 |
| PYHLSSQYSRT | 13 |
| WHLSSQYSRT | 14 |
| YHLSSQYSRT | 15 |
| WHLSSQYSR | 16 |
| YHLSSQYSR | 17 |

In some embodiments, the CTP comprises the amino acid sequence HLSSQWSR (SEQ ID NO: 18). In certain embodiments, the CTP comprising the amino acid sequence HLSSQWSR (SEQ ID NO: 18) is about 8 to 12 amino acids in length, or about 8 to 10 amino acids in length; examples of such CTPs include, but are not limited to, CTPs having the sequences listed in Table 4.

TABLE 4
Sequences of CTPs comprising HLSSQWSR (SEQ ID
NO: 18), and that are conjugated to the nucleic
acid molecule.

| CTP SEQUENCE | SEQ ID NO |
|---|---|
| APWHLSSQWSRT | 19 |
| PWHLSSQWSRT | 20 |
| WHLSSQWSRT | 21 |
| WHLSSQWSR | 22 |

In preferred embodiments, the CTP has the amino acid sequence APWHLSSQYSRT (SEQ ID NO: 6).

The CTP may be manufactured by methods known in the art, for example, by fluorenylmethyloxycarbonyl (FMOC) chemistry.

The nucleic acid molecule and CTP may be linked by a covalent bond or a non-covalent bond, optionally via one or more linker molecules. In embodiments in which the nucleic acid molecule and CTP are linked by a covalent bond, optionally via one or more linker molecules, the covalent bond may be selected from a peptide bond, thioester bond, thioether bond, carbamate bond, or combination thereof. In certain embodiments, the nucleic acid molecule and CTP are linked by a disulfide bond. Such bonds can be created according to methods generally and well known in the art.

In embodiments in which the nucleic acid molecule and CTP are linked via one or more linker molecules, the linker may be a peptide. The peptide may comprise a length of about 1 to 50 amino acids, or about 1 to 20, or about 1 to 10, or about 1 to 5 amino acids. In certain embodiments, the linker may comprise a cleavage site, such as an enzymatic or chemical cleavage site, which can release the CTP from the nucleic acid molecule.

Further discussion and other examples of CTPs that can be used in the present invention is provided in U.S. Pat. No. 9,249,184 and U.S. Patent Publication No. 2021/0206805, which are incorporated herein by reference.

In some embodiments, the nucleic acid molecule may be synthesized with a terminal thiol group and then conjugated to the side amine group of the CTP via a mono-dithio-bis-maleimidoethane (DTME) intermediate. Thus, in some embodiments, the conjugate comprises the nucleic acid molecule, the CTP, and DTME.

In certain aspects, the present invention provides a composition, e.g., a pharmaceutical composition, comprising the conjugate comprising a nucleic acid molecule and a CTP. Preferably, the composition comprises one or more carriers, diluents, excipients, or other additives. For example, the composition can comprise one or more bulking agents (e.g., dextran 40, glycine, lactose, mannitol, trehalose), one or more buffers (e.g., acetate, citrate, histidine, lactate, phosphate, Tris), one or more pH adjusting agents (e.g., hydrochloric acid, acetic acid, nitric acid, potassium hydroxide, sodium hydroxide), and/or one or more diluents (e.g., water, physiological saline). The pH of the composition is preferably between about 3.0 and 8.0. In one embodiment, the pH is between about 3.5 and 6.5, or between about 5.0 and 7.5.

The pharmaceutical composition of the invention may be prepared by methods known in the art. For example, the methods may comprise admixing the conjugate and a pharmaceutically acceptable carrier to prepare the composition.

An aspect of the present invention relates to the conjugate, or pharmaceutical composition thereof, as described herein, for use in any of the methods of the present invention described herein.

The invention further provides pharmaceutical packs or kits comprising one or more containers filled with the conjugate or pharmaceutical composition thereof. Optionally associated with such container(s) can be a notice in the form prescribed by a governmental agency regulating the manufacture, use or sale of pharmaceuticals or biological products, which notice reflects approval by the agency of manufacture, use or sale for human administration.

### EXAMPLES

### Example 1: Effect of miRNAs In Vitro Model of Heart Failure

A study was conducted to evaluate the effects of miRNA17, miRNA20a, miRNA93, and miRNA106a on an in vitro model of heart failure.

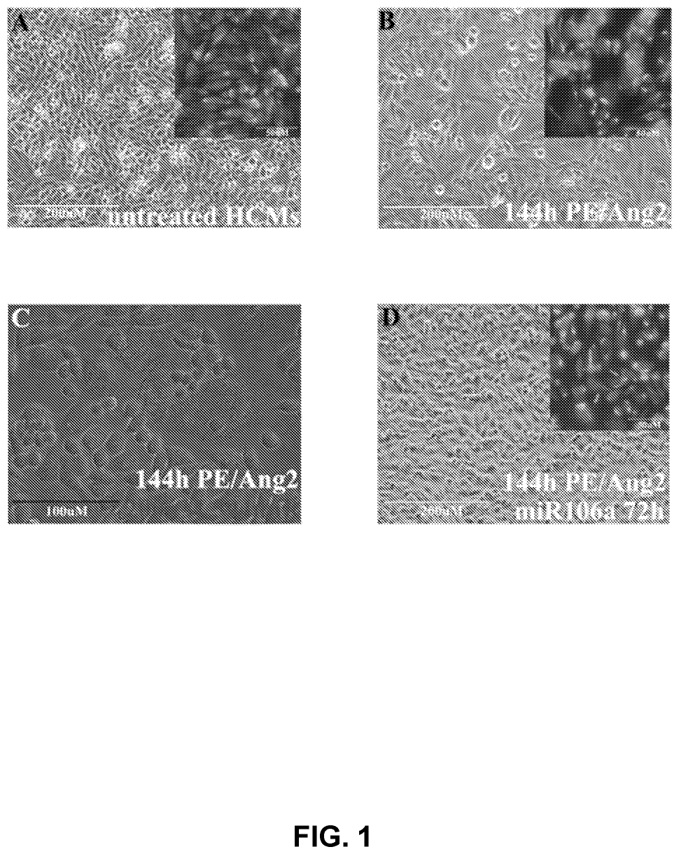

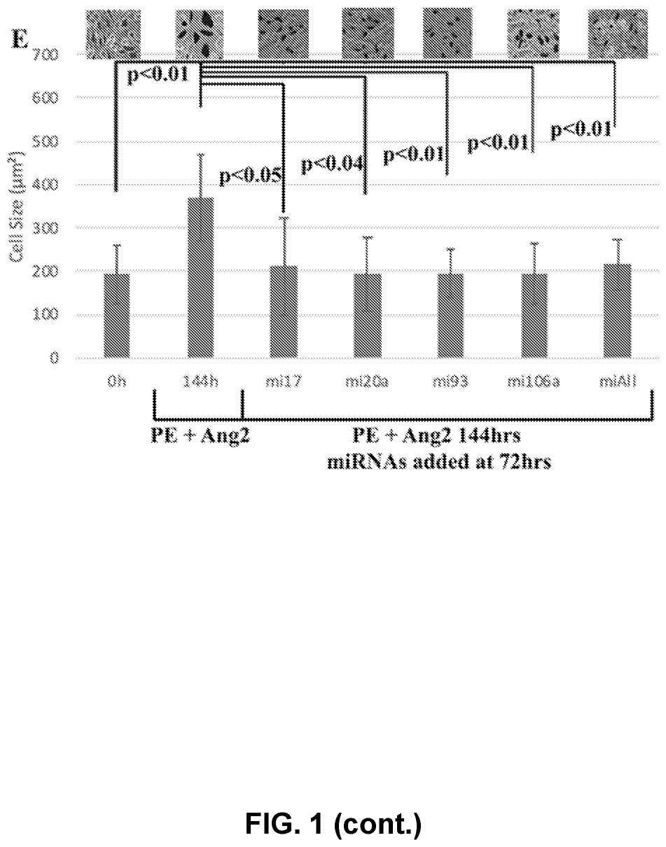

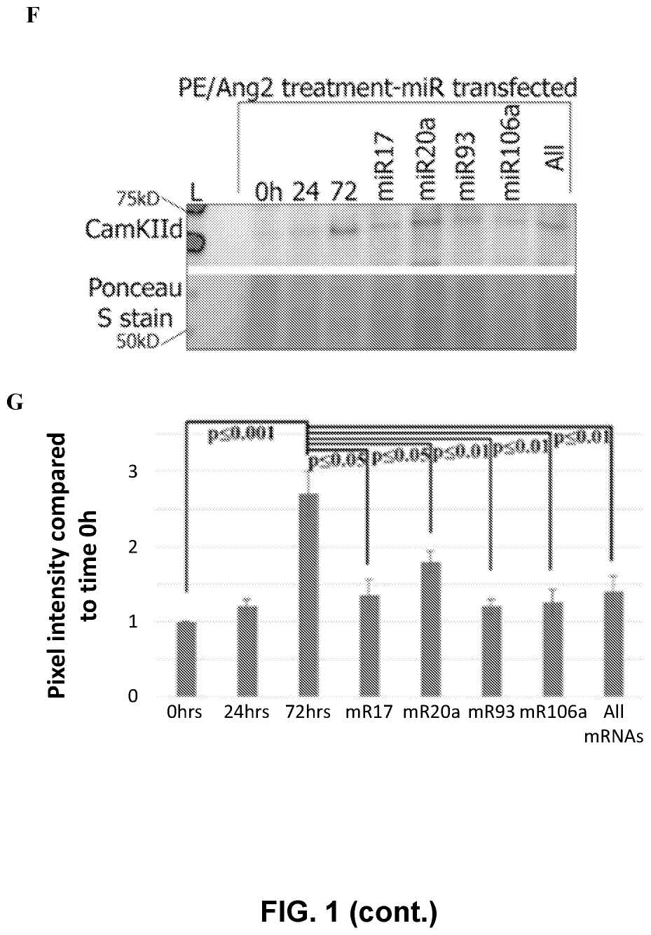

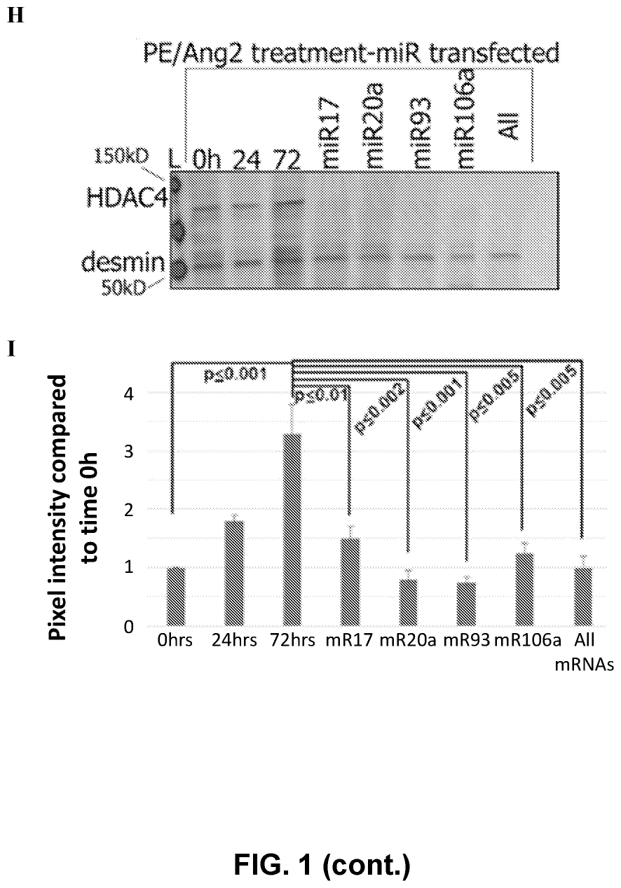

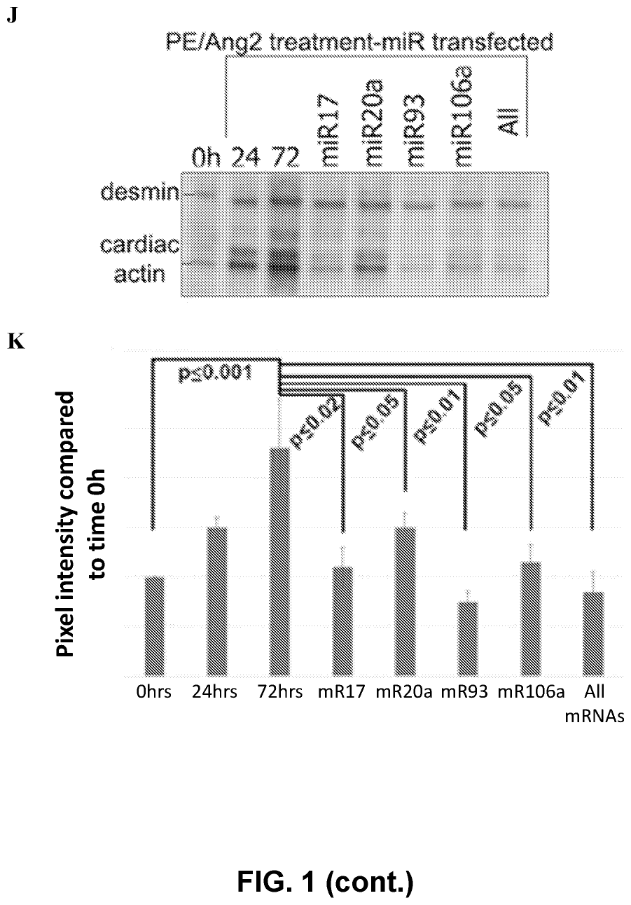

Human cardiomyocytes (HCMs) were subjected to 200 nM phenylephrine (PE) and 10 nM angiotensin 2 (Ang2) to induce hypertrophy (initial stages of heart failure). The HCMs were treated with PE/Ang2 for 24, 48, 72, 96, or 144 hours. Using TransIT-X2 transfection reagent, 200 nM of each miRNA, or in combination, was introduced into PE/Ang2-treated HCMs, followed by analyses of hypertrophic morphology and heart failure gene/protein expression. As shown in FIG. 1, Panels A-E, the miRNAs significantly rescued/reverted hypertrophic HCMs back to their normal phenotypic dimensions even in the presence of PE/Ang 2 treatment.

Western blot analyses of CaMKIIδ (FIG. 1, Panels F and G) and HDAC4 (FIG. 1, Panels H and I) showed that each of miRNA17, miRNA20a, miRNA93, and miRNA106a suppressed, within 72 hours, CaMKIIδ and HDAC4 overexpression that had been induced by PE/Ang2. These results suggest that CaMKIIδ and HDAC4 are targets for miRNA17, miRNA20a, miRNA93, and miRNA106a.

In addition, treatment with each miRNA resulted in less cardiac actin expression (FIG. 1, Panels J and K). Cells that increase in size require more cardiac actin, and cells that decrease in size require less cardiac actin. Therefore, the decrease in cardiac actin expression shown in the results reflects the cells' reversion back to normal size.

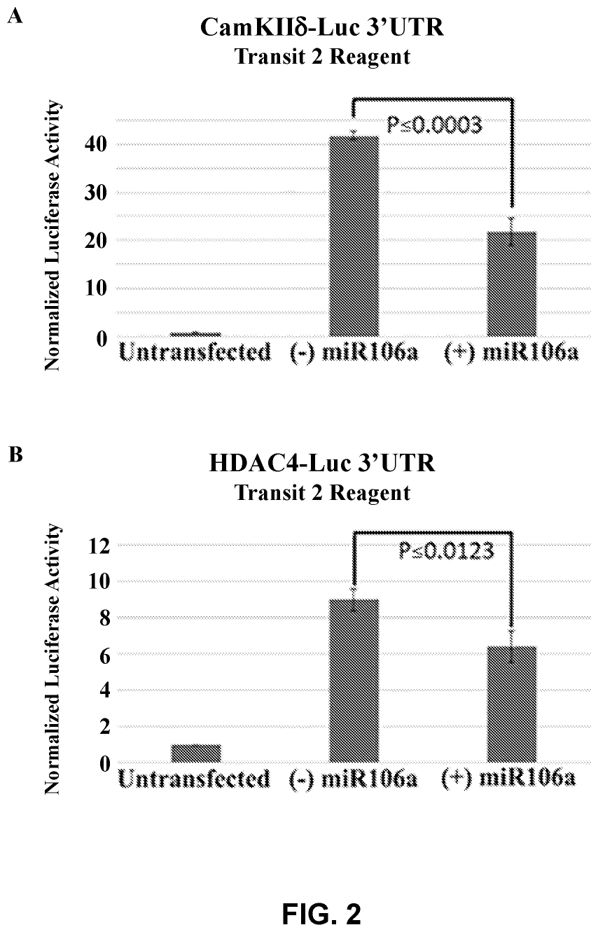

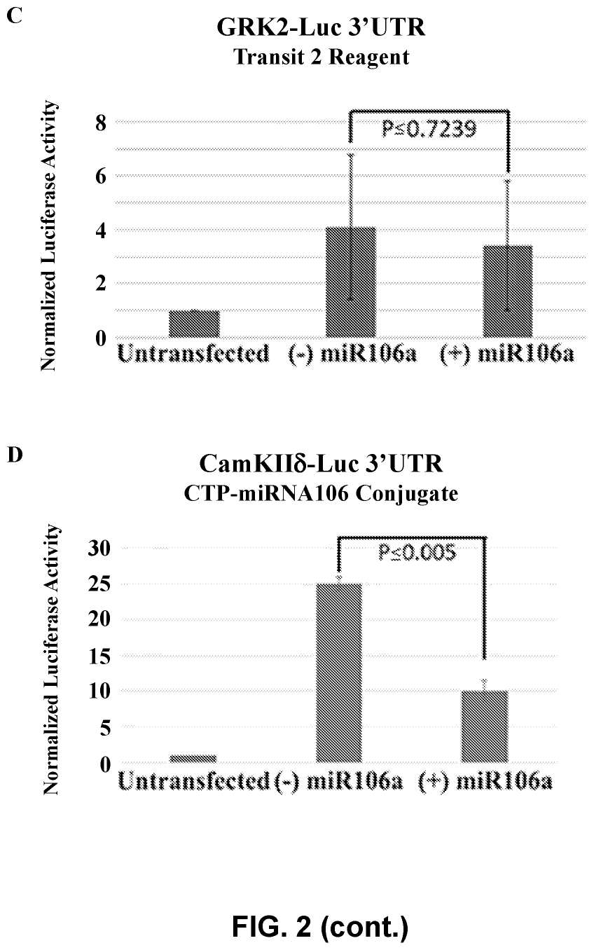

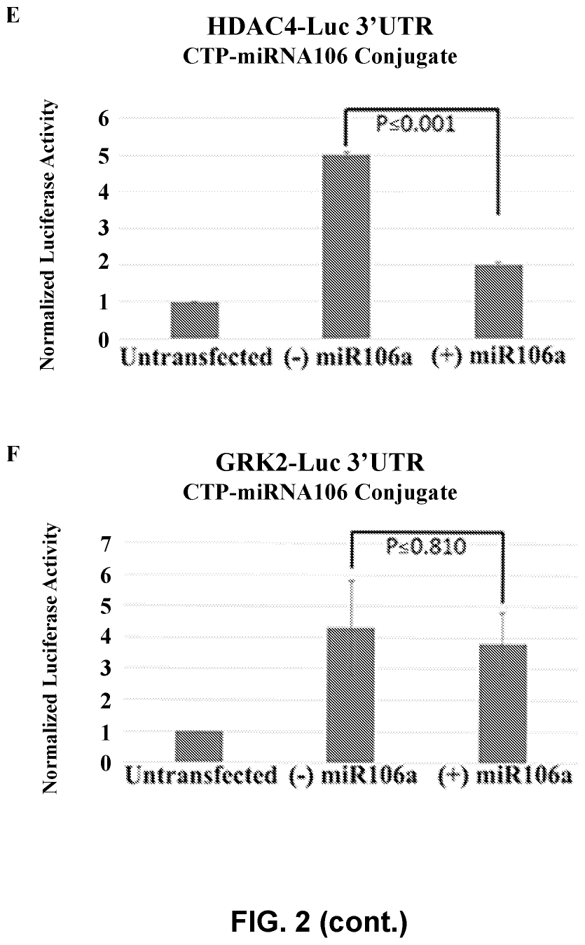

As confirmation, full length 3′UTRs for CaMKIIδ, HDAC4, and GRK2 were fused to firefly/renilla luciferase constructs. 3′UTR-luciferase assays showed that miRNA106a targets the 3′UTRs of both CaMKIIδ and HDAC4 (FIG. 2, Panels A and B, respectively). The 3′UTR for GRK2, which was used as a control because it does not have a target sequence for miRNA106a, showed no change in luciferase expression (FIG. 2, Panel C).

### Example 2: Delivery of CTP-Conjugated microRNAs to HCMs

A study was conducted to evaluate the delivery of miRNA106a as a conjugate with a CTP (“CTP-miRNA106a conjugate”). The conjugate comprised miRNA106a, with a thiol modified 5′ end, linked through a disulfide bond to the CTP, which was labeled with Cy5.5. The CTP was a peptide having the amino acid sequence of APWHLSSQYSRT (SEQ ID NO: 6).

The CTP-miRNA106a conjugate were studied in HCMs, HEK293 cells (i.e., a high-transfection-efficiency cell line), and other cardiac cell types (e.g., cardiac fibroblasts, endothelial cells). The effects of introducing the miRNA106a as the CTP-miRNA106a conjugate were also compared to the effects of delivering miRNA106a using TransIT-X2 transfection reagent.

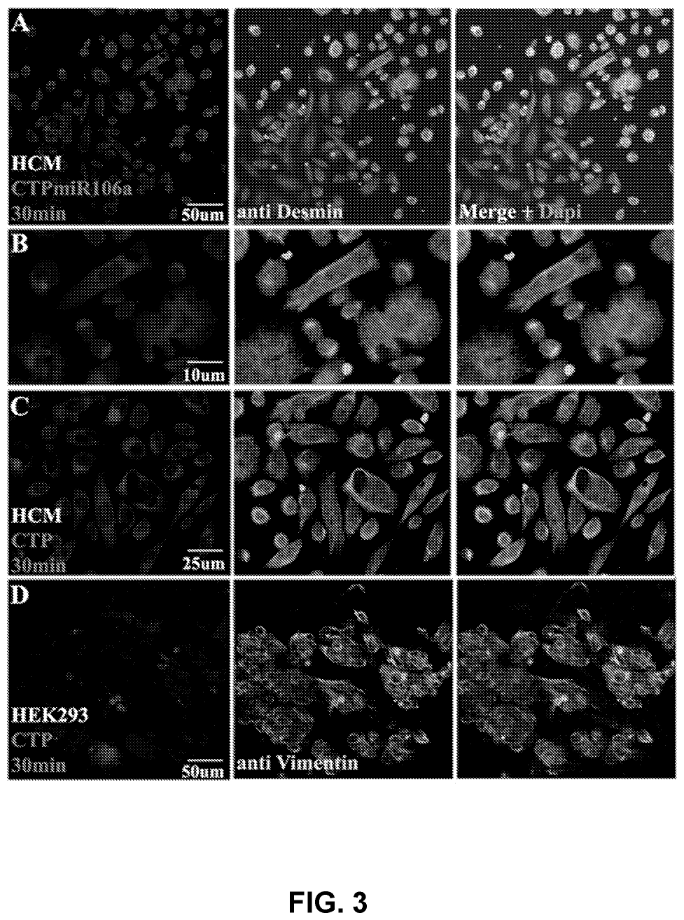

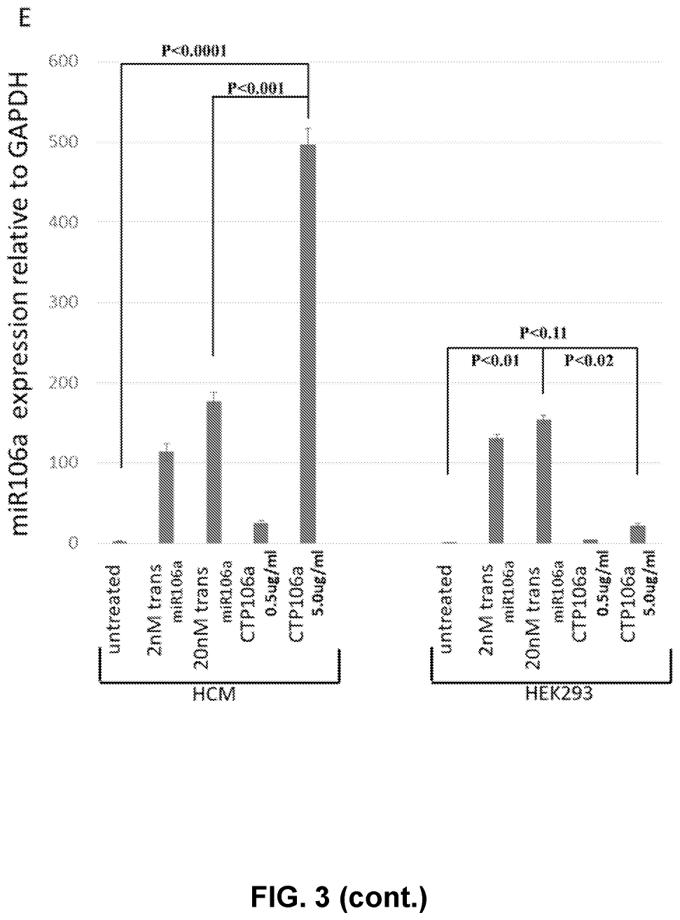

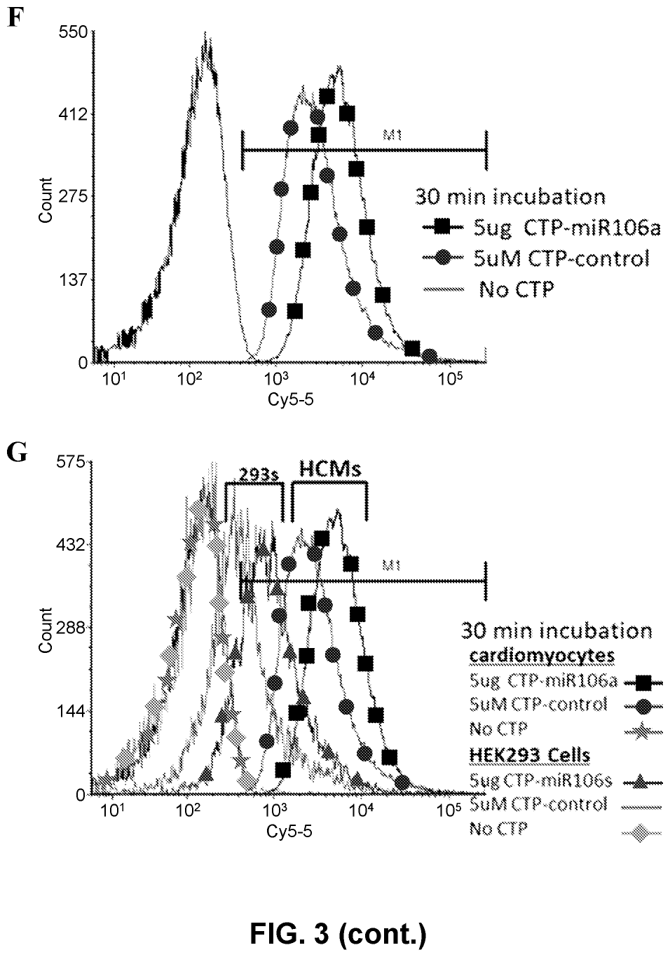

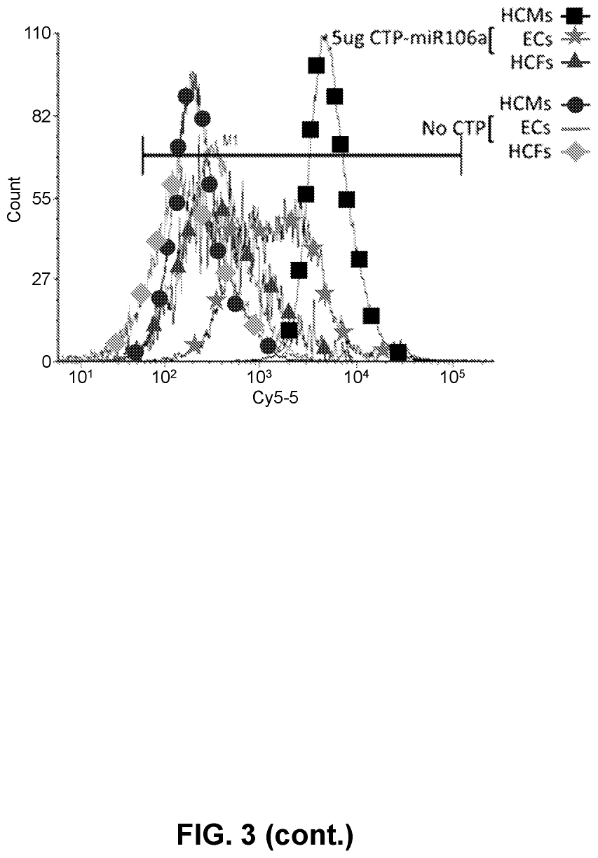

The CTP-miRNA106a conjugate, which was linked to a fluorescent Cy5.5 marker targeted HCMS and not HEK293 cells, even though HEK293 cells were specifically chosen as a control because they are considered easy to transfect (FIG. 3, Panels A-D). Molecular analysis using quantitative RT-PCR demonstrated that 5 μg/ml of the CTP-miRNA106a conjugate, which is equivalent to 200 nM of miRNA106, delivered ˜2.5× more miRNA106a to cardiomyocytes than transfecting 200 nM miRNA106a using the TransIT-X2 transfection reagent (FIG. 3, Panel E). FACS analyses further confirmed the specificity of delivery of CTP-miRNA106a conjugate to HCMs (FIG. 3, Panels F and G). Virtually no increase in miRNA106a was detected in non-cardiac HEK293 cells treated with CTP-miRNA106a. In addition, of the three basic types of cells found in the heart—cardiomyocytes, cardiac fibroblasts, and endothelial cells—a small signal overlap of the CTP-miRNA106a conjugate was only detectable within endothelial cells (FIG. 3, Panel H).

The luciferase assay was performed on HCMs subjected to the CTP-miRNA106a conjugate, without using TransIT-X2 transfection reagent. The results of the assay showed that the miRNA106a delivered by CTP targeted both CaMKIIδ and HDAC4 3′UTRs (see FIG. 2, Panels D and E, respectively), resulting in significant decrease in luciferase expression, but such a reduction was not observed in GRK2 (FIG. 2, Panel F). This result indicated that miRNA106a was physically free and bioactive, and was processed correctly in the RNA inhibitory silencing complex (RISC) in order for miRNA to directly interact with its target mRNA and suppress translation of luciferase.

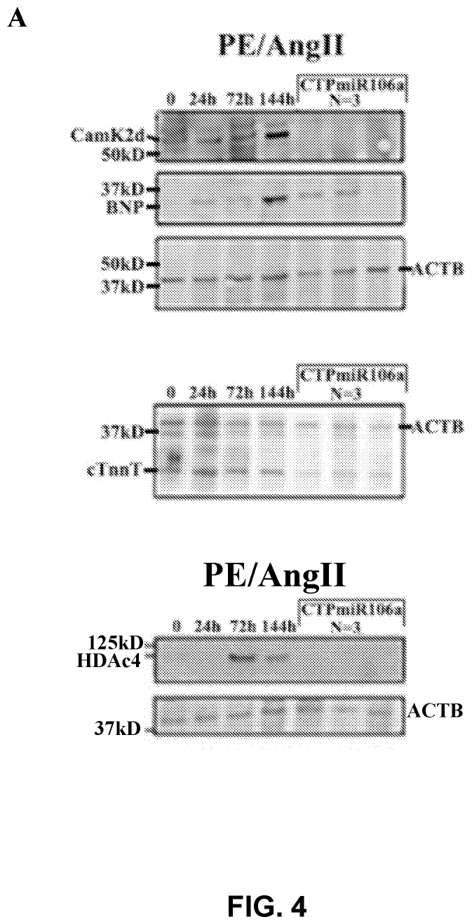

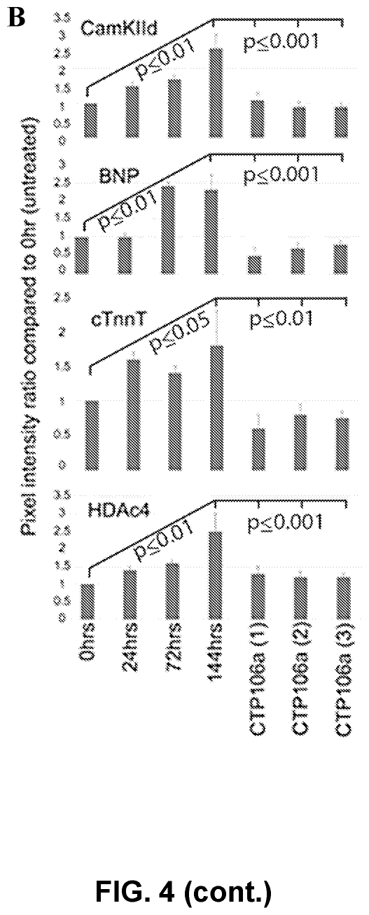

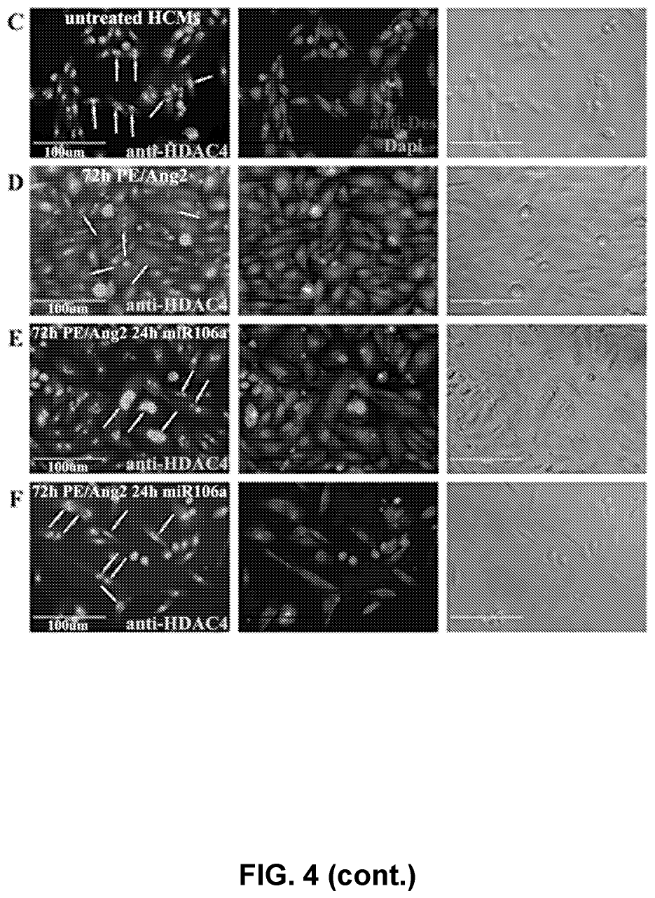

Further evaluation using Western analysis demonstrated that the miRNA106a delivered by CTP reversed PE/Ang2-induced hypertrophic responses. FIG. 4, Panels A and B show that PE/Ang2 induced expression of CamKIIδ and HDAC4, while the CTP-miRNA106a conjugate suppressed and returned the expression of these proteins to normal (untreated) levels. The CTP-miRNA106a conjugate suppressed translation of each protein, as confirmed in three separate experiments. A significant difference was observed between each of the CTP-miRNA106a conjugate-treated set and PE/Ang2-treated HCMs after 144 hours, and no significant difference was observed between each of the CTP-miRNA106a conjugate-treated HCMs and untreated HCMs.

Western analysis was also used to assess the CTP-miRNA106a conjugate's impact on downstream targets (and markers for hypertrophy) of CaMKIIδ- and HDAC4-signaling in HCMs subjected to PE/Ang2 treatment. The results show that both BNP and cTnnT increased in response to PE and Ang2, however, they both subsequently returned to baseline (untreated) levels in response to the CTP-miRNA106a conjugate (see FIG. 4, Panels A and B). BNP and cTnnT are not targets for miRNA106a.

HDAC4 localization was also rescued by the CTP-miRNA106a conjugate. HDAC4 is nuclear in untreated HCMs (see FIG. 4, Panel C), but CaMKIIδ phosphorylation promotes HDAC4 movement into the cytoplasm (see FIG. 4, Panel D) (Hohl et al., 2013). Transfection of miRNA106a using the CTP-miRNA106a conjugate and TransIT-X2 results in HDAC4 re-entry into the nucleus (see FIG. 4, Panel E and F, respectively). Notably, compared to untreated cells (see FIG. 4, Panel C), PE/Ang2 caused a marked increase in cell size (see FIG. 4, Panel D), which was reversed after the treatment with the conjugate (see FIG. 4, Panel E and F, respectively).

### Example 3: Effect of CTP-Conjugated microRNAs on GRK2 Signaling

The effects of miRNA17, miRNA20a, miRNA93, and miRNA106a on GRK2 signaling were assessed in HCMs treated with PE/Ang2. GRK2 has been identified to cause heart failure when mis-expressed.

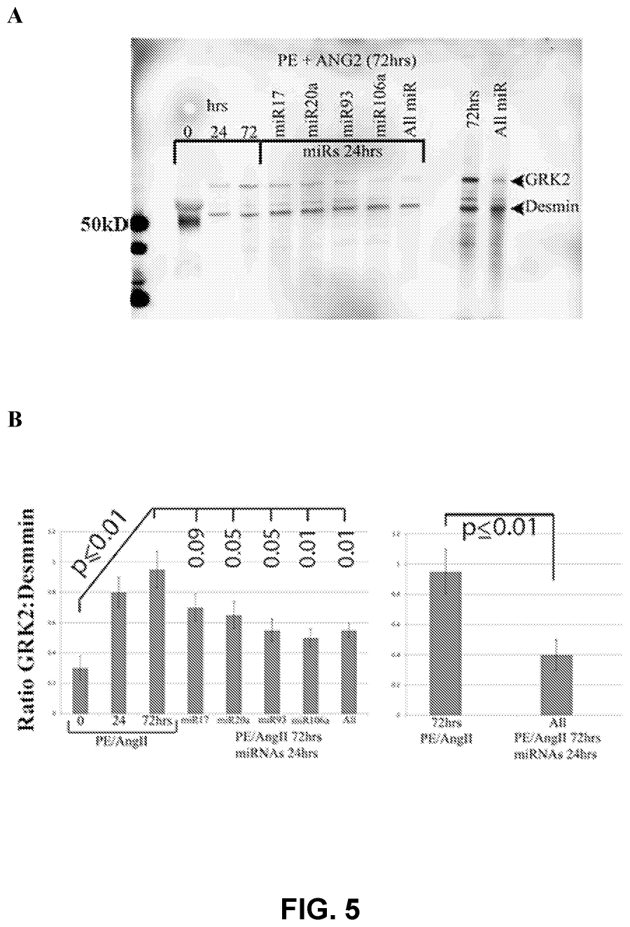

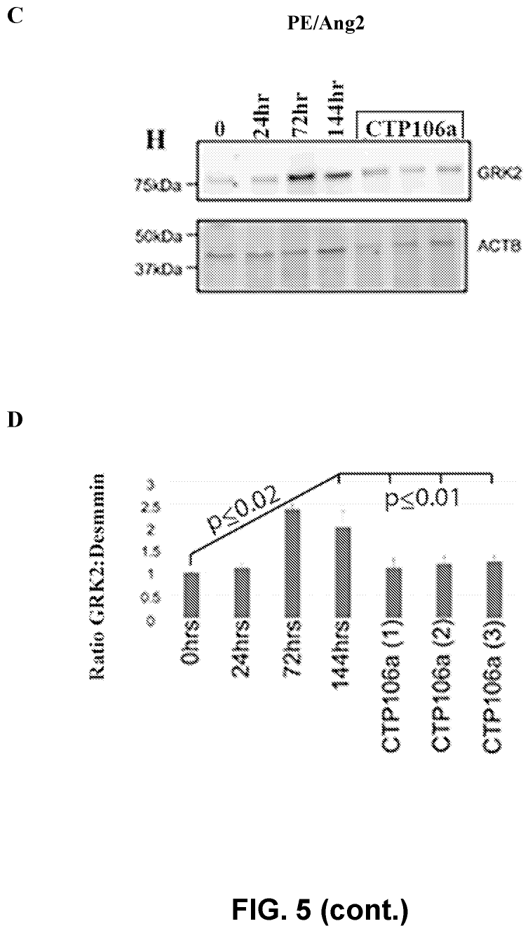

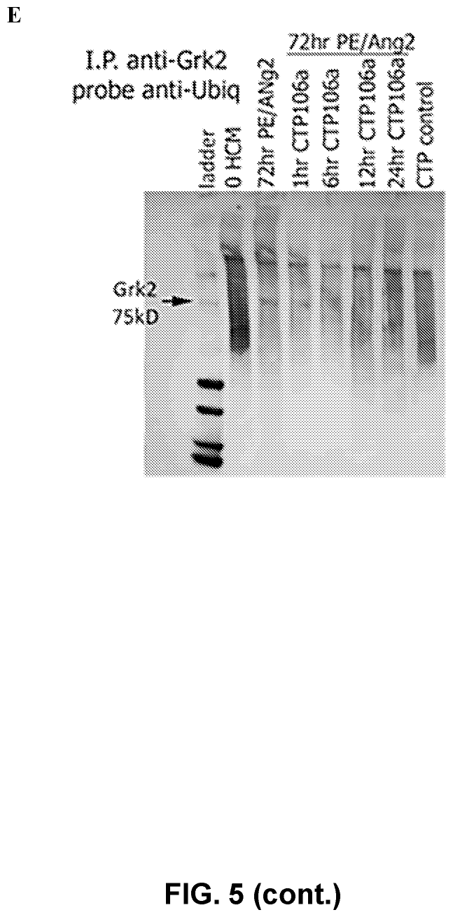

The HCMs were subjected to 72 hours of PE/Ang2 treatment, and were subsequently treated with miRNA17, miRNA20a, miRNA93, or miRNA106a, or with the CTP-miRNA106a conjugate. The results show that PE/Ang2 caused an increase in GRK2 expression, but GRK2 expression returned to baseline levels after transfection of each miRNA (FIG. 5, Panels A and B) or after treatment with the CTP-miRA106a conjugate (FIG. 5, Panels C and D).

GRK2 is not a target of miRNA17, miRNA20a, miRNA93, and miRNA106a, but some evidence suggests that GRK2 phosphorylation by CaMKIIδ prevents ubiquitination resulting in excess GRK2 (Gambardella et al., 2020). To identify if ubiquitination levels of GRK2 were involved with these changes, an immunoprecipitation assay was performed. The results showed that PE/Ang2 treatment (which increased CaMKIIδ expression) led to a marked decrease in ubiquitin staining by 72 hours of PE/Ang2 treatment, but addition of the CTP-miRNA106a conjugate over time increased levels of ubiquitinated GRK2 (see FIG. 5, Panel E).

### Example 4: Effect of CTP-Conjugated microRNAs on Mitochondrial Health

There is evidence that miRNA106a may cause cardiac hypertrophy by targeting and suppressing Mfn2, a mitochondrial membrane protein involved in maintaining mitochondrial structure (Guan et al., 2016).

The effects of miRNA106a on mitochondrial health was assessed using the mitochondrial membrane potential (MMP) probe JC-1 staining. Healthy MMP is observed as JC-1 forms J-aggregates that fluoresce red (˜594 nm) while unhealthy MMP is recognized as green (˜488 nm) JC-1 monomers.

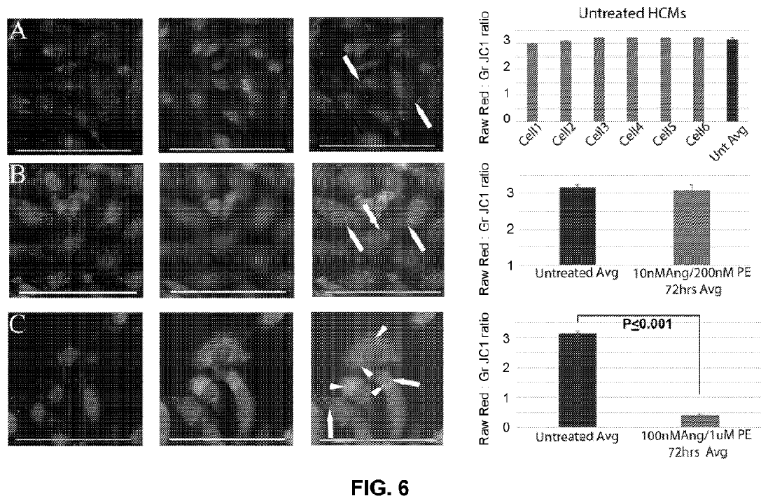

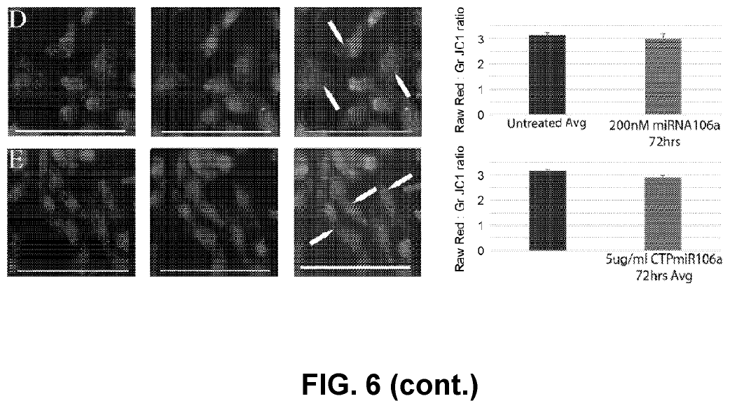

Untreated HCMs showed virtually no green puncta fluorescence-only red (FIG. 6, Panel A). The ratios of red:green pixel intensity of six cells averaged ˜3.1 (FIG. 6, Panel F).

HCMs treated with 10 nM Ang2 and 200 μM PE for 72 hours resulted in few unhealthy (green puncta) mitochondria (FIG. 6, Panel B). There was no significant difference in red:green ratios between untreated and treated (FIG. 6, Panel G). HCMs treated for 72 hours with ten times the amount of Ang2/PE normally used to induce hypertrophy resulted in significantly more green (unhealthy) mitochondria (FIG. 6, Panel C) when compared to untreated HCMs.

Transfection of 200 nM miRNA106a and culturing for 72 hours shows mostly red puncta (FIG. 6, Panel D) and no significant difference in red:green ratios (FIG. 6, Panel I) when compared to untreated. Notably, HCMs did not appear to grow in size (become hypertrophic) in response to 200 nM miRNA106a transfection. Green MMPs were observed if ten times the amount of the CTP-miRNA106a conjugate, as compared to the amounts discussed in Examples 2 and 3, was used for at least 72 hours.

Using 5 μg/ml of the CTP-miRNA106a conjugate to deliver miRNA106a to HCMs, followed by incubation for 72 hours, resulted in only red mitochondria (FIG. 6, Panel E). There was no difference in red:green ratios when compared to untreated HCMs (FIG. 6, Panel J).

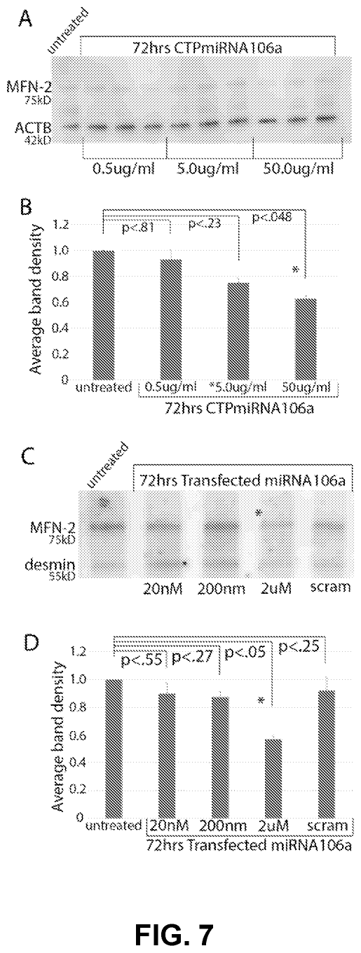

Western blots were used to identify Mfn2 protein expression in response to miRNA106a. HCMs cultured in three increasing dosages of the CTP-miRNA106a conjugate resulted in a significant decrease in Mfn2 expression only at 50 μg/ml, which is ten times the amount needed for reducing CamKIIδ, HDAC4, and GRK2 that was demonstrated in Examples 2 and 3 (FIG. 7, Panel A). FIG. 7, Panel B shows the band density for three separate experiments at each dose. HCMs cultured in three increasing transfection concentrations of miRNA106a for 72 hours showed lowered expression of Mfn2 at 2 μM, which is significant (FIG. 7, Panels C and D).

### Example 5: Effect of the CTP-miRNA106a Conjugate on NfκB Pathway

The nuclear factor kappa-B (NfκB) pathway can exacerbate heart failure by activating genes involved with inflammation, such as the interleukins, IL-1β, IL-6, and tumor necrosis factor-alpha (TNF-α) (Stansfield et al., 2014). Both protein kinase C (PKC) and CamK2d have been shown to activate the NfkB pathway by phosphorylating the NfkB inhibitory protein Ika, which is then degraded, enabling the NfkB transcription factor to enter the nucleus and activate genes. Thus, the effect of the CTP-miRNA106a conjugate on the NfκB pathway was studied.

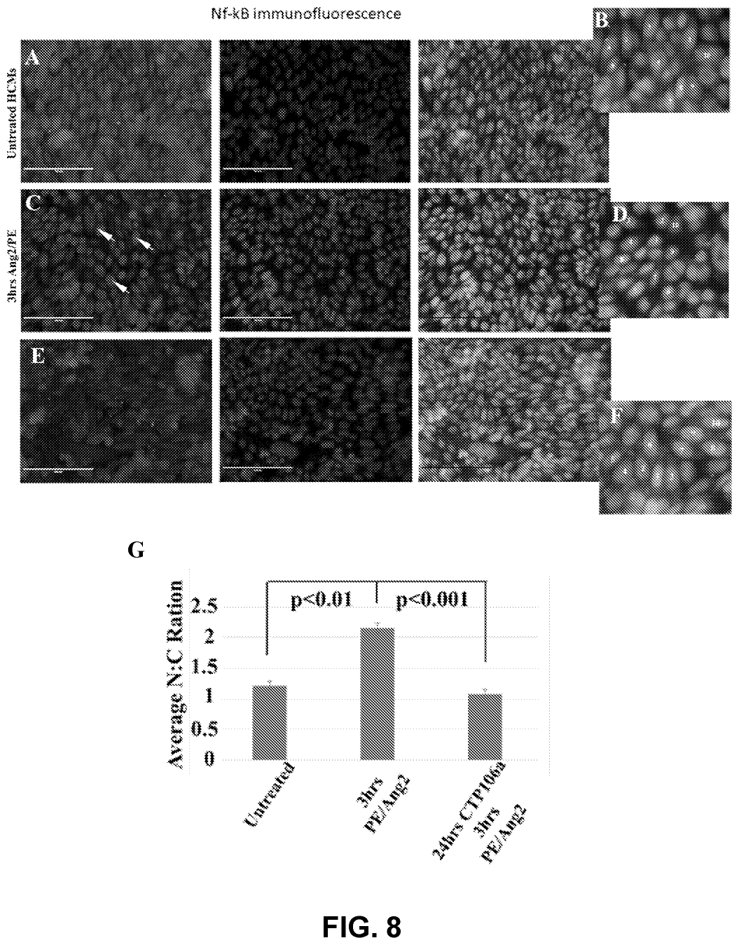

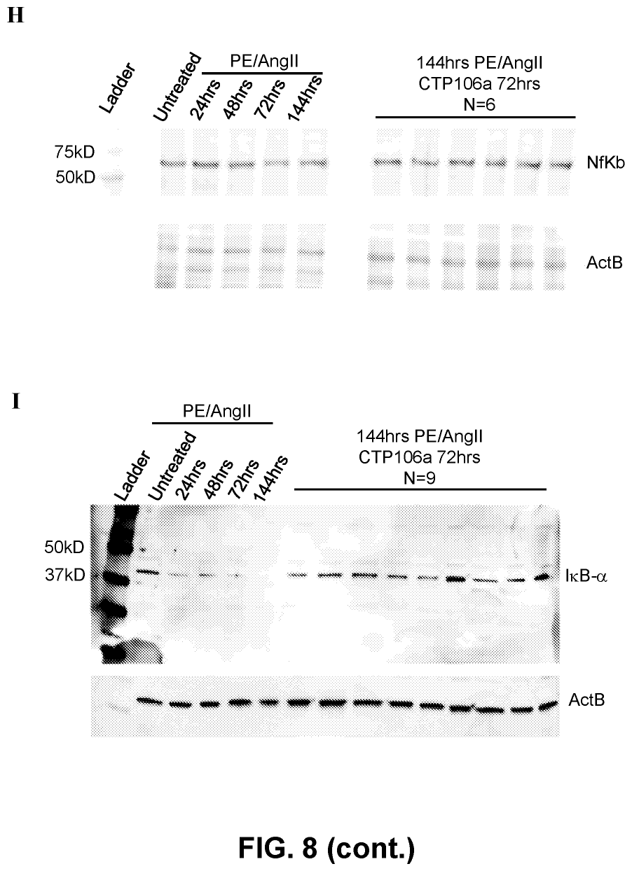

Untreated HCMs were compared to HCMs treated with Ang2/PE or HCMs pretreated with the CTP-miRNA106a conjugate for 24 hrs and then treated with Ang2/PE. In untreated HCMs, NfkB diffused within cells, localizing to both the cytoplasm and nuclei (FIG. 8, Panels A and B). In HCMs treated with Ang2/PE, after only three hours NfκB localized to the nucleus of the cells (FIG. 8, Panels C and D, showing distinct nuclear staining of NfκB). However, this localization was prevented by pre-treatment of the HCMs with the CTP-miRNA106a conjugate (FIG. 8, Panel E and F, identifying cells with cytoplasmic and nuclear staining and showing few cells with distinct nuclear translocation of NfκB; FIG. 8, Panel G, which quantifies the merged staining and determined that Ang2/PE induced NfκB translocation into the nucleus while the CTP-microRNA106a prevented this translocation). In addition, western blot analysis showed that NfκB staining was relatively equal intensity at time 0 to 144 hrs of Ang2/PE treatment, and that the CTP-microRNA106a pretreatment did not alter NfκB staining intensity (FIG. 8, Panel H). Further, Ika was degraded over time when cells were cultured in Ang2/PE, but subjecting cells to the CTP-miRNA106a conjugate prevented such Ika degradation (FIG. 8, Panel I).

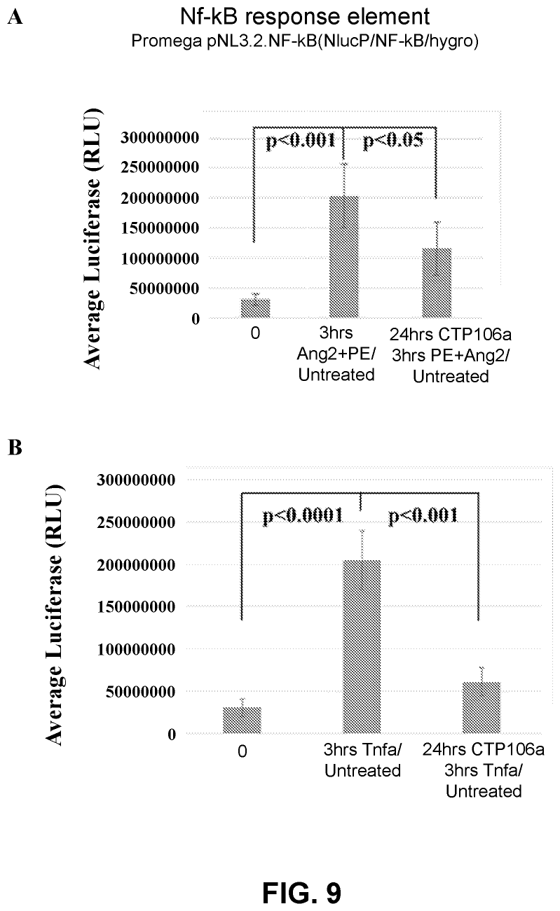

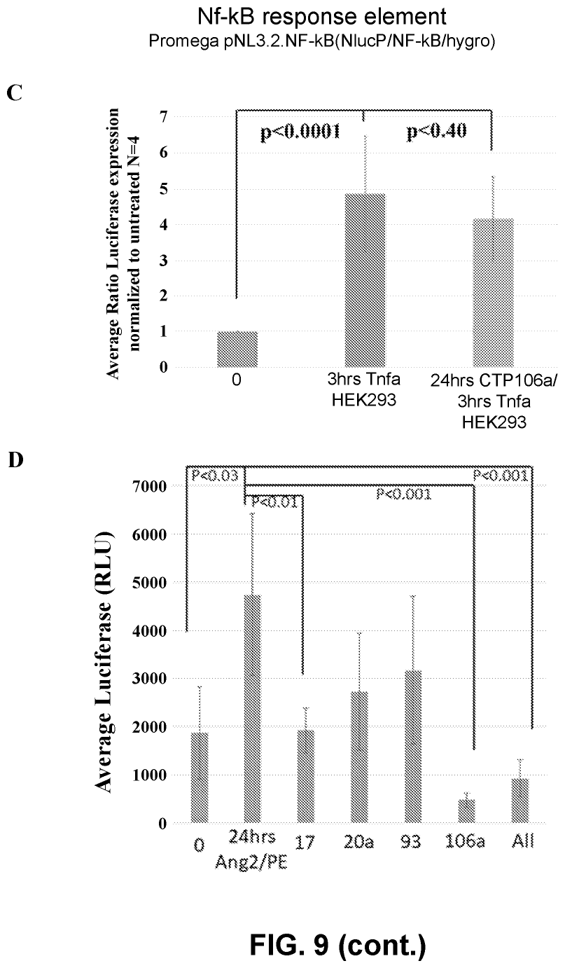

Once in the nucleus NfκB binds to an NfκB response element to initiate transcription of downstream genes. To study how the CTP-miRNA106a conjugate can impact this effect, a plasmid containing an NfκB response element driving luciferase production (Promega, Inc) was used to generate HCMs expressing this NfκB luciferase response element. Treatment with Ang2/PE for three hours resulted in a significant increase in luciferase expression, and such increase was reduced by 24-hour pretreatment with the CTP-miRNA106a conjugate (FIG. 9, Panel A). This effect of the CTP-miRNA106a conjugate was surprisingly demonstrated when, as a control, HCMs were treated with TNF-α, a known activator of NfκB; treatment with TNF-α for three hours resulted in a significant increase in luciferase expression; but pretreatment with the CTP-miRNA106a conjugate for 24 hours significantly decreased luciferase expression (FIG. 9, Panel B). As a further control, HEK293 cells were used to show that the CTP-miRNA106a conjugate did not decrease the luciferase expression induced by TNF-α in a non-cardiomyocyte cell type, which supports the finding that the CTP-miRNA106a conjugate targets cardiomyocytes and not cells of kidney origin (FIG. 9, Panel C). Moreover, the effect of miRNA106a on impacting Ang2/PE-induced luciferase expression in HCMs was compared to miRNA17, miRNA20a, and miRNA93. The miRNAs were transfected into HCMs expressing the NfκB luciferase response element treated with Ang2/PE for 24 hours and, while all four miRNAs lowered luciferase expression, miRNA106a and miRNA17 suppressed luciferase expression significantly (FIG. 9, Panel D).

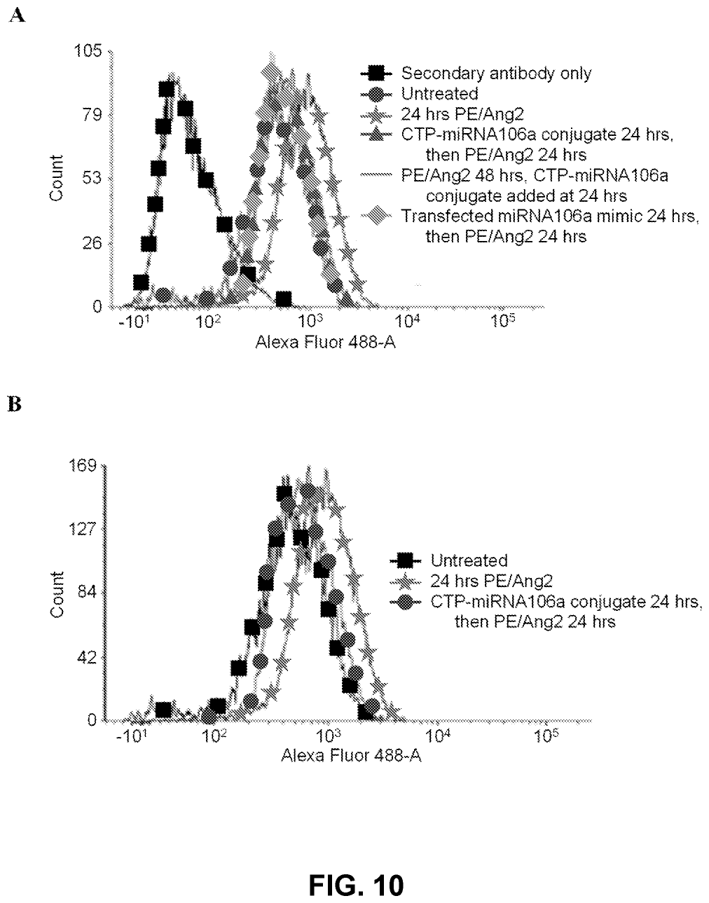

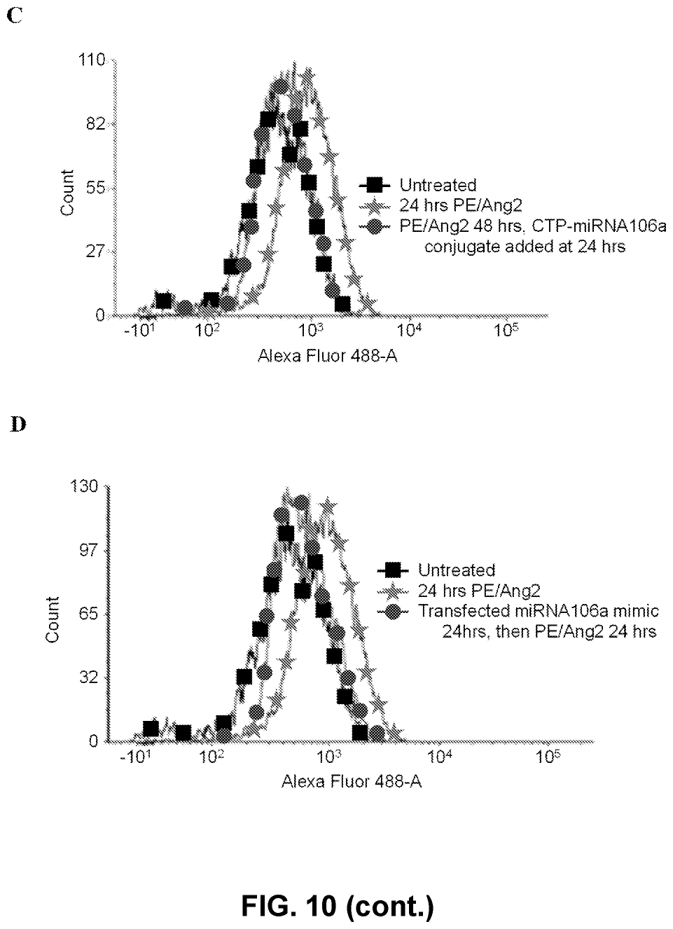

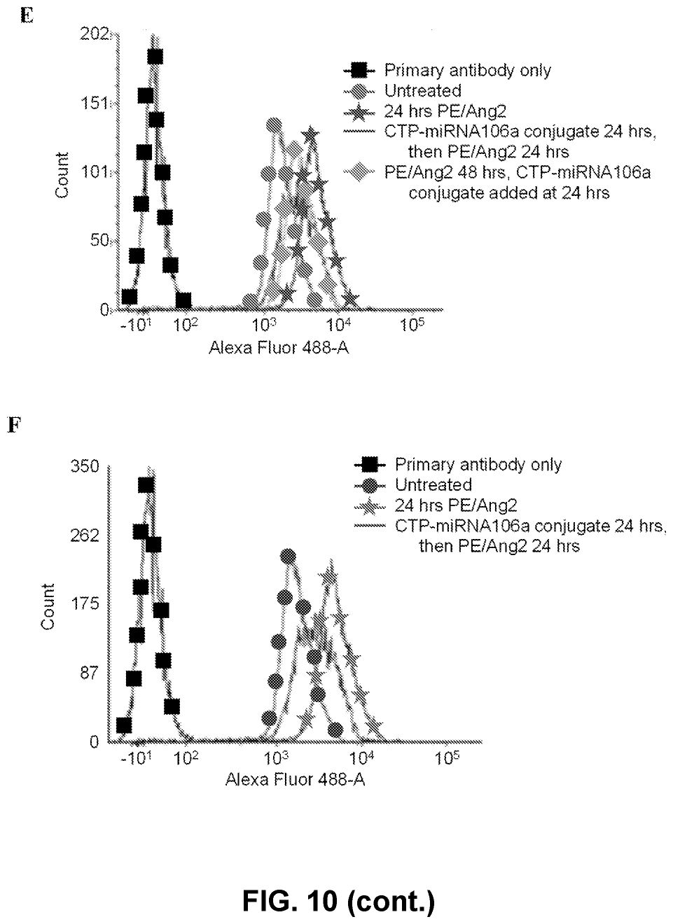

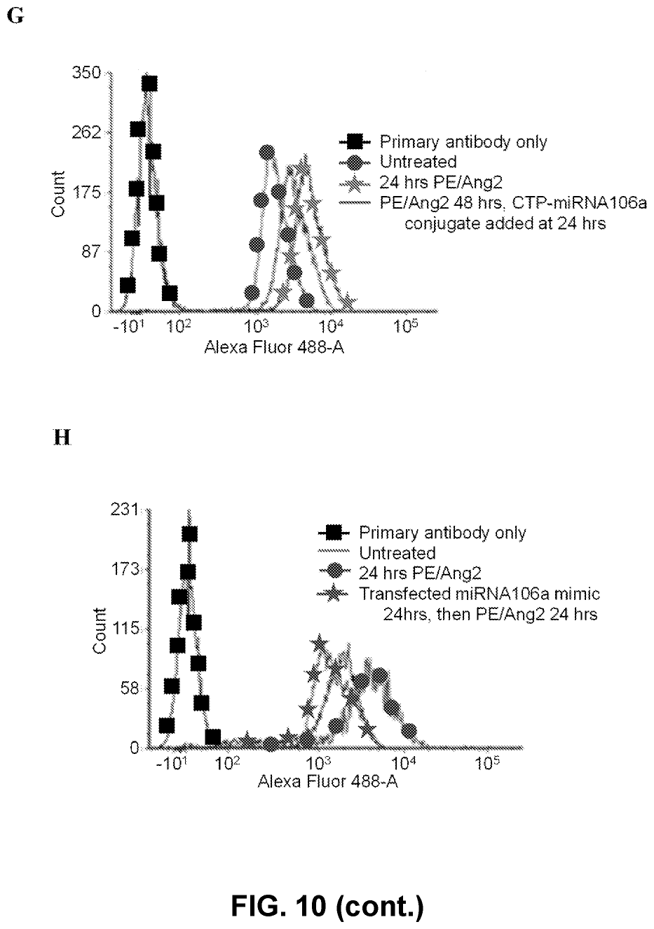

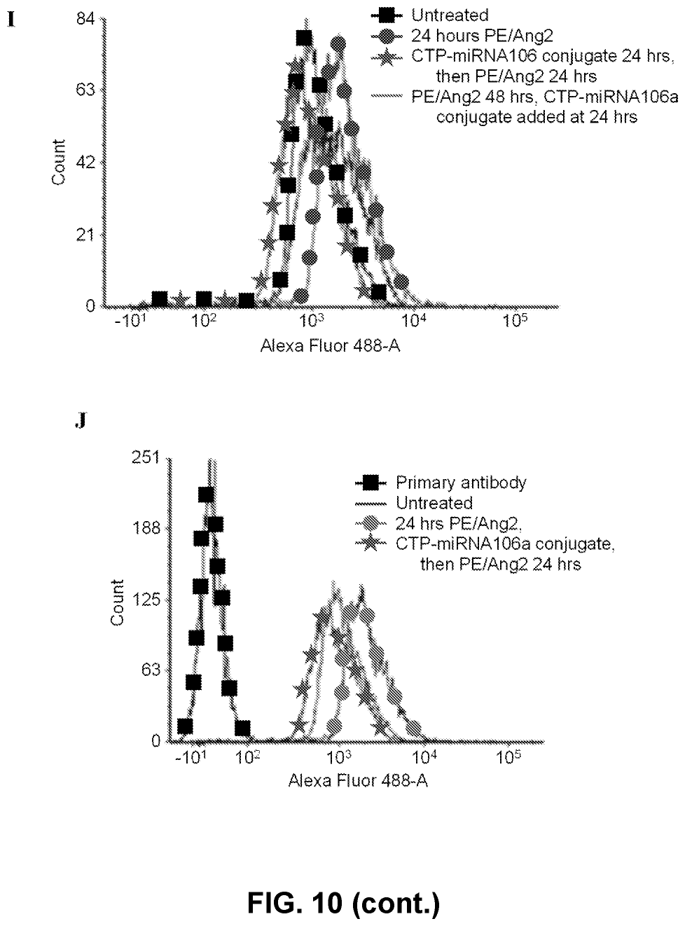

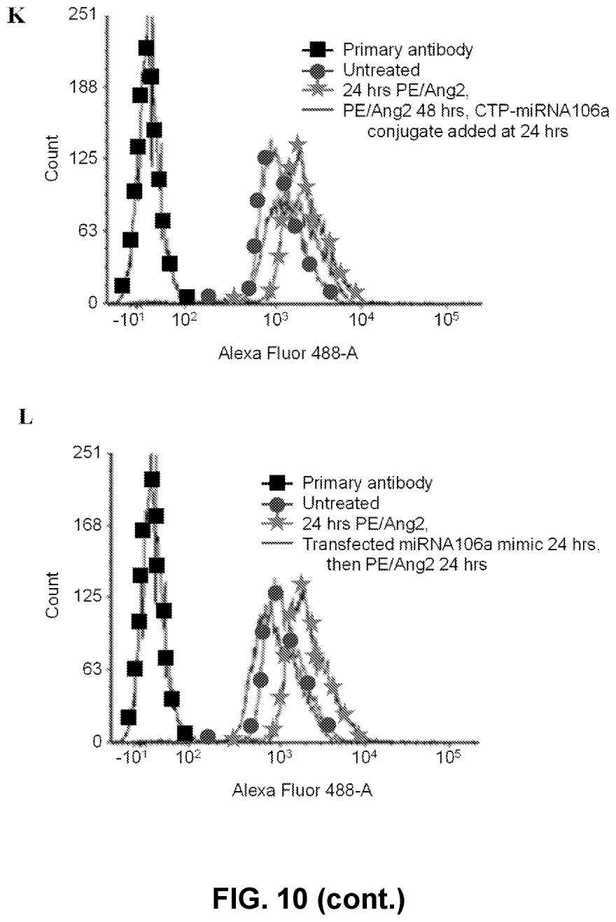

To identify if activation of the NfκB pathway by Ang2/PE led to expression of genes that augment hypertrophy and heart failure, HCMs treated with Ang2/PE or treated with both Ang2/PE and the CTP-miRNA106a conjugated were compared to untreated control HCMs by FACS. Using an IL-1ß antibody linked to fluorescein, FACS analysis showed that Ang2/PE increased the number of cells expressing IL-1β, but treatment with the CTP-miRNA106a conjugate-either prior to or after the Ang2/PE treatment-reversed the number of cells expressing IL-1ß back to normal, untreated levels (FIG. 10, Panels A-C). As a control, miR106a (without CTP) was transfected into HCMs followed by treatment with Ang2/PE, and these cells also showed a reversal of IL-1ß expression back to normal, untreated levels (FIG. 10, Panel D). IL-6 (FIG. 10, Panels E-H) and TNF-α (FIG. 10, Panels I-L), both of which are also genes activated by NfκB, showed very similar results as IL-1β. In summary, the CTP-miRNA106a conjugated suppressed NfκB signaling activated by Ang2/PE.

### Example 6: Effect of the CTP-miRNA106a Conjugate on PLCβ1 Pathway

The phospholipase C beta 1 (PLCβ1) gene pathway can induce cardiac hypertrophy through overactive PKC and calcium release. Thus, the effect of the CTP-miRNA106a conjugate on the PLCβ1 gene pathway was studied.

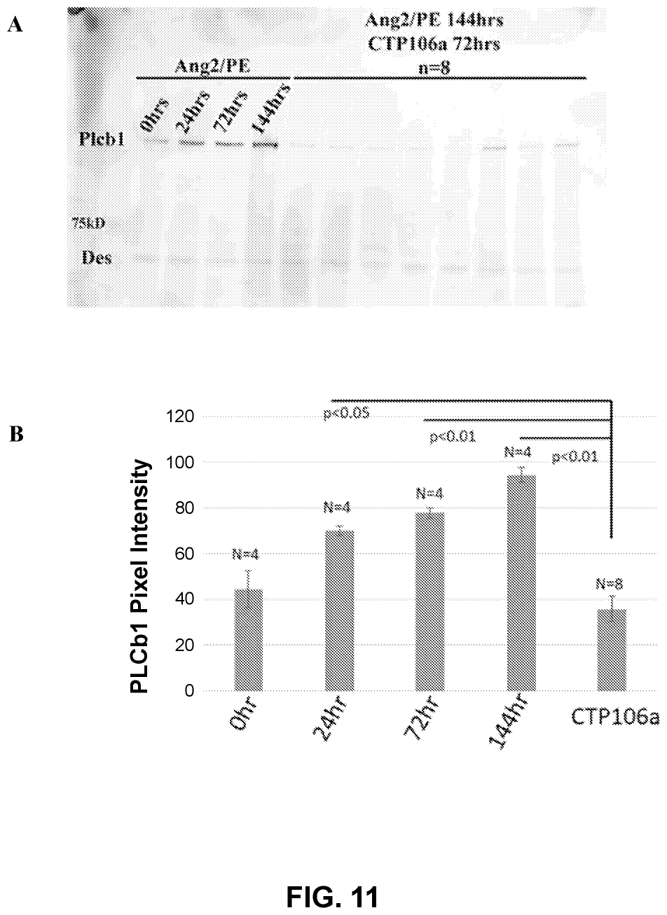

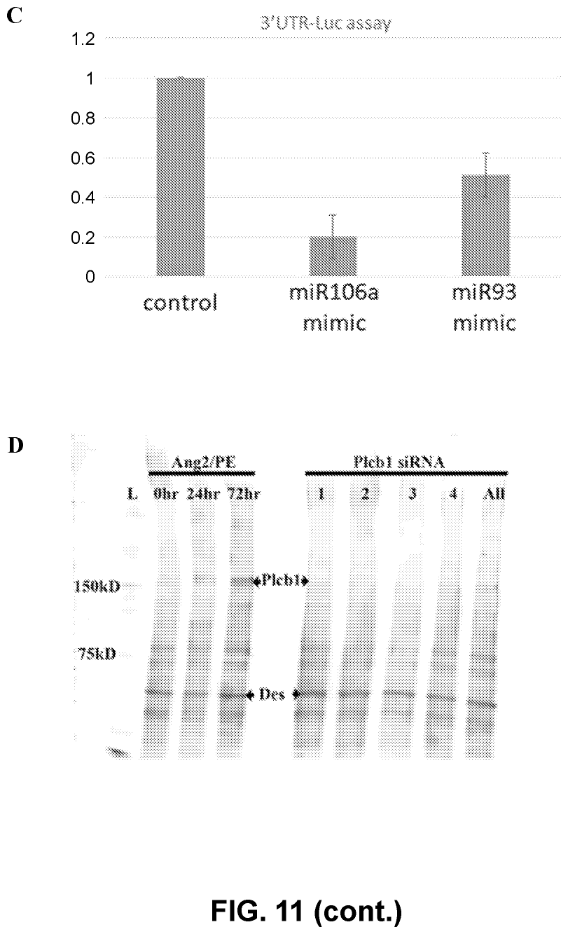

In HCMs incubated in Ang2/PE over time, there was in increase expression of PLCβ1, but addition of the CTP-microRNA106a conjugate for 72 hours resulted in a significant decrease in PLCβ1 expression as compared to expression levels after 24 to 144 hours of Ang2/PE incubation, even returning PLCβ1 expression to 0-hour (untreated) levels (FIG. 11, Panels A and B). To confirm PLCβ1 is a target of miRNA106a, a plasmid containing a CMV promoter driving luciferase linked to the 3′UTR of PLCβ1 was transfected into HEK293 cells, followed 24 hours later by transfection of miRNA 106a or miRNA93 (same seed sequence). miRNA106a significantly decreased luciferase activity by ˜80%, confirming PLCβ1 as a target for MiRNA106a, and miRNA93 decreased luciferase activity by ˜50%. (FIG. 11, Panel C). In addition, siRNAs was used to knock down PLCβ1 as a general control for loss of expression. Four siRNAs verified by Dharmacon to target PLCβ1 all were able to knock-down PLCβ1 at 200 nM (FIG. 11, Panel D). These results provide molecular and cellular confirmation that miRNA106a targets PLCβ1, and show that Ang2/PE causes increased expression of PLCβ1 over time. It is speculated that increased expression of PLCβ1 is most likely contributing to the heart failure phenotype through its persistent cleavage of phosphatidyl inositol bisphosphate (PIP2) into diacyl glycerol (DAG) and inositol triphosphate (IP3), both of which overtly activate PKC.

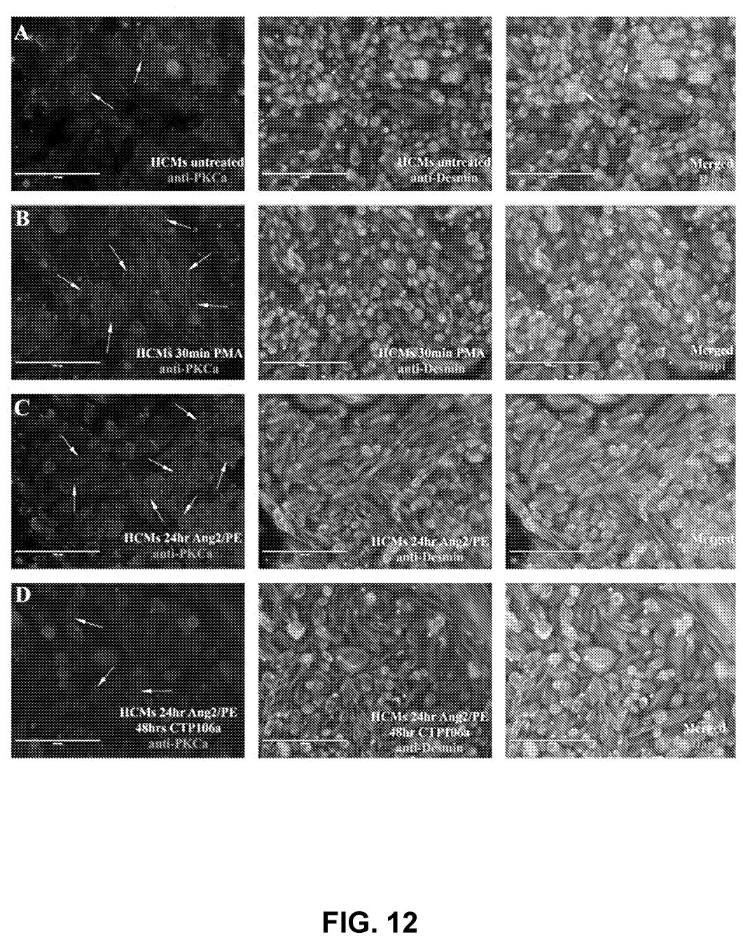

One potential mechanism of increased PLCβ1 expression is increased PKC activity. Thus, PKC localization was studied in treated HCMs. In untreated HCMs, few cells showed membrane localization of PKC (FIG. 12, Panel A). In HCMs treated with PMA, a synthetic activator of PKC, PKC was localized to the plasma membrane after 30 minutes (FIG. 12, Panel B). In HCMs treated with Ang2/PE, PKC was translocated to the plasma membrane (FIG. 12, Panel C). However, the additional treatment of CTP-miRNA106a conjugate in the Ang2/PE-treated HCMS reversed PKC translocation to the plasma membrane (FIG. 12, Panel D). Together, these results show that Ang2/PE induction of PKC to translocate to the plasma membrane was inhibited by the CTP-miRNA106a conjugate.

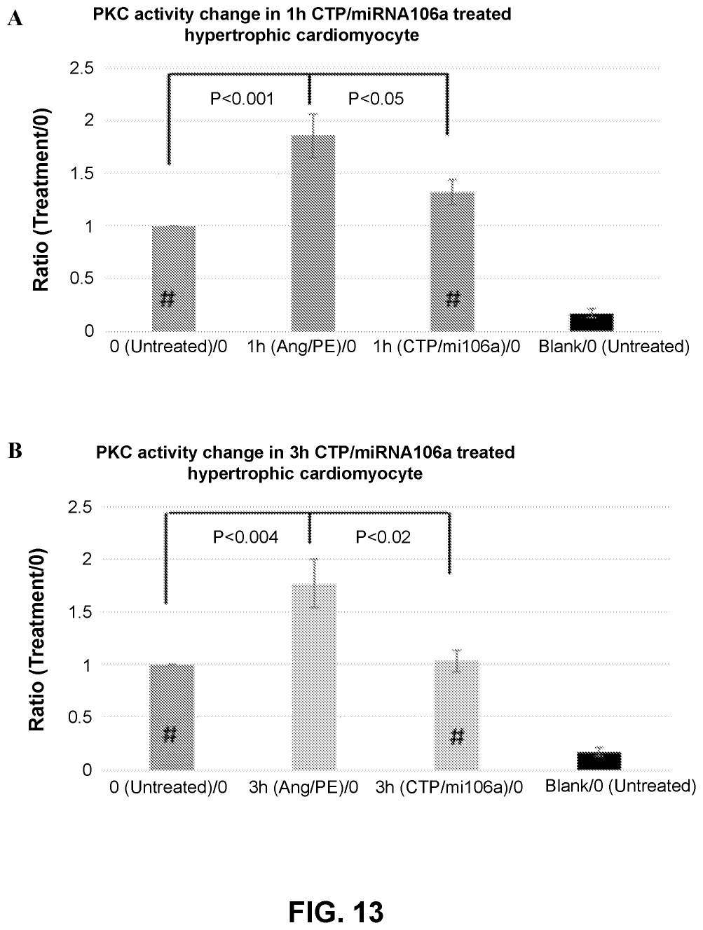

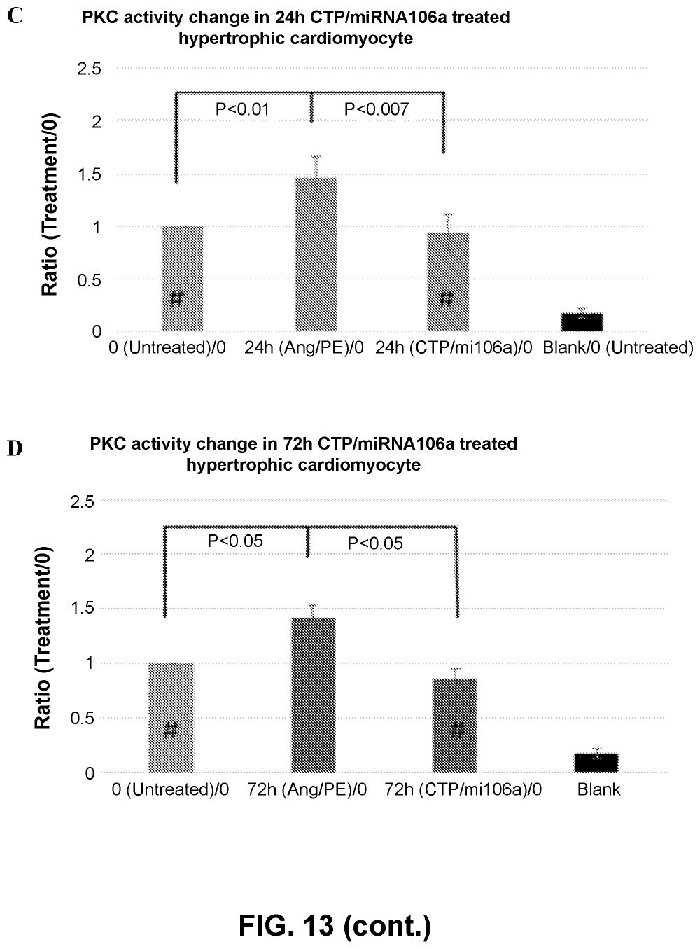

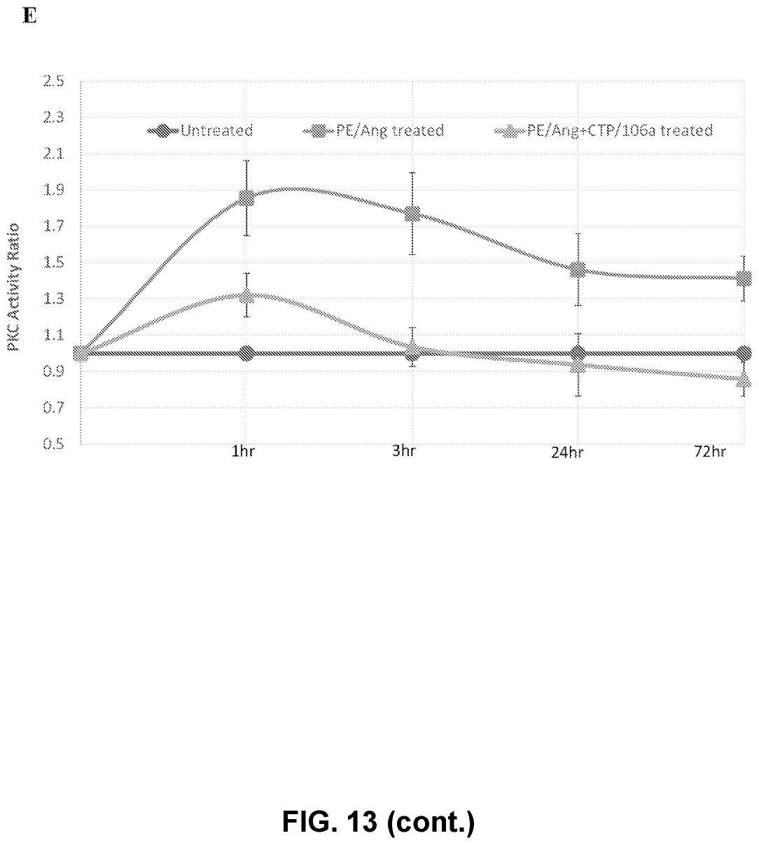

To confirm that Ang2/PE increased PKC activity and to verify the CTP-microRNA106a conjugate could suppress this activation, a PKC-specific activity assay (ADI-EKS-420A) from Enzo Life Science Inc. was employed on untreated HCMs, HCMs treated with Ang2/PE, HCMs treated with Ang2/PE and the CTP-microRNA106 conjugate. Normalizing PKC activity ratios to untreated HCMs (0), one-hour treatment with Ang2/PE resulted in significant elevation of PKC activity, however, this elevated activity could be prevented by treatment with the CTP-microRNA106a conjugate (FIG. 13, Panel A). This same pattern was demonstrated when HCMs were treated for three hours (FIG. 13, Panel B), 24 hours (FIG. 13, Panel C), and 72 hours (FIG. 13, Panel D). Comparing PKC activity at each of these time points, there was a spike in activity of Ang2/PE at one hour, followed by a decrease and eventual plateau; the CTP-microRNA106a conjugate significantly suppressed this spike and plateau of PKC activity (FIG. 13, Panel E). Based on these results, the CTP-microRNA106a conjugate seems to be capable of reducing PKC expression in response to over stimulation by Ang2/PE.

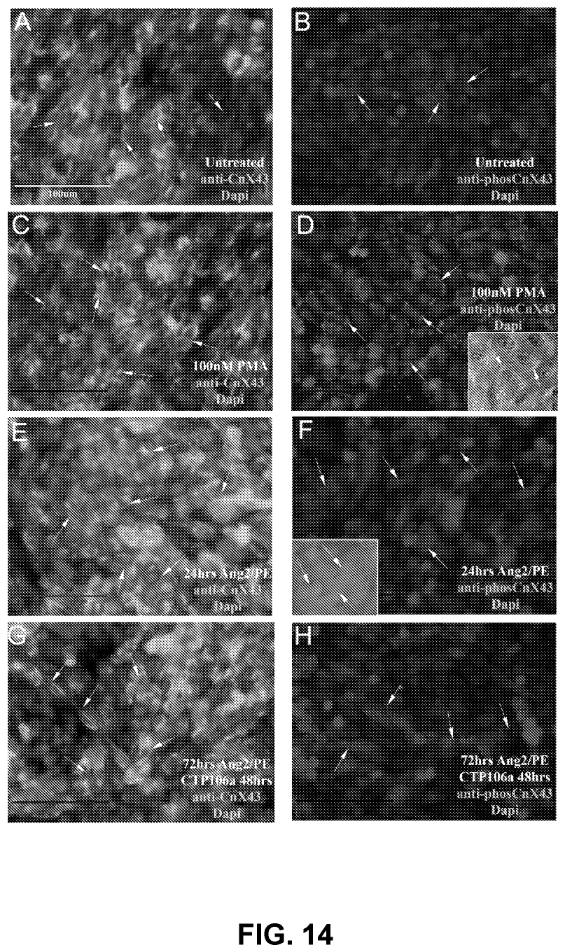

Gap junction protein Connexin 43 (CNX43) is a known downstream target of PKC activity in cardiomyocytes. Thus, the effects of Ang2/PE and the CTP-microRNA106a on CNX43 in HCMs were studied. CNX43 is phosphorylated on serine 386 by PKCα. In untreated HCMs, gap junctions are prevalent (FIG. 14, Panel A), although only a few junctions are positive for phosphorylation of CNX43 (FIG. 14, Panel B). In HCMs treated with PMA as a control, the phosphorylation patterns are more robust (FIG. 14, Panels C and D). Phosphorylated CNX43 becomes widespread as HCMs are treated with Ang2/PE for 24 hours (FIG. 14, Panels E and F). However, when HCMs are treated with Ang2/PE for 72 hours and the CTP-microRNA106a conjugate for 48 hours, the gap junctions remain intact but the presence of phosphorylated CNX43 decreases (FIG. 14, Panels G and H). These results indicate that the CTP-microRNA106a conjugate can decrease Ang2/PE-induced CNX43 phosphorylation.

### Example 7: Effect of the CTP-miRNA106a Conjugate in Mouse Model of Heart Failure

To study the effects of the CTP-miRNA106a conjugate in vivo, the CTP-miRNA106a conjugate was administered to mice that were induced to experience heart failure through osmotic pumps that delivered Ang2 and isoproterenol (Ang2/Iso). The study involved three cohorts of mice that differed in the amount of Ang2/Iso delivered and the timing of the administration of the CTP-miRNA106a conjugate. The first cohort comprised mice that were delivered saline (n=2), 500 μg/kg/d Ang2 and 30 ng/kg/d Iso (n=2), and 500 μg/kg/d Ang2 and 30 ng/kg/d Iso along with injections of 10 mg/kg of the CTP-miRNA106a conjugate at weeks 5, 6, 7, and 8 post-Ang2/Iso delivery (n=2). The second cohort comprised mice that were delivered saline (n=3), 2 mg/kg/d Ang2 and 30 ng/kg/d Iso (n=4), and 2 mg/kg/d Ang2 and 30 ng/kg/d Iso along with injections of 10 mg/kg of the CTP-miRNA106a conjugate at weeks 3 and 4 post-Ang2/Iso delivery (n=2). The third cohort comprised mice that were delivered saline (n=1), received no delivery of saline or Ang2/Iso (n=2); 1.5 mg/kg/d Ang2 and 30 ng/kg/d Iso (n=2), and 1.5 mg/kg/d Ang2 and 30 ng/kg/d Iso along with injections of 10 mg/kg of the CTP-miRNA106a conjugate at weeks 2, 3, 4, 5, 6, and 7 post-Ang2/Iso delivery (n=2).

M-mode, ultrasound baseline measurements were acquired from C57/BL6 mice at week 0, followed by implantation of osmotic pumps containing the Ang2/Iso or saline. Ultrasound measurements were performed to identify ejection fraction (EF) levels <45%, which signified entry into heart failure. The CTP-miRNA106a conjugate was then injected via tail vein at the specified weeks in addition to weekly ultrasound measurements.

The three- and four-dimensional ultrasonic imaging was performed utilizing the VisualSonics Vevo 3100. The 4D-Strain software allows to assess cardiac function across the mouse models and helps to inform the impacts of the CTP-miRNA106a conjugate to reverse or suppress heart failure.

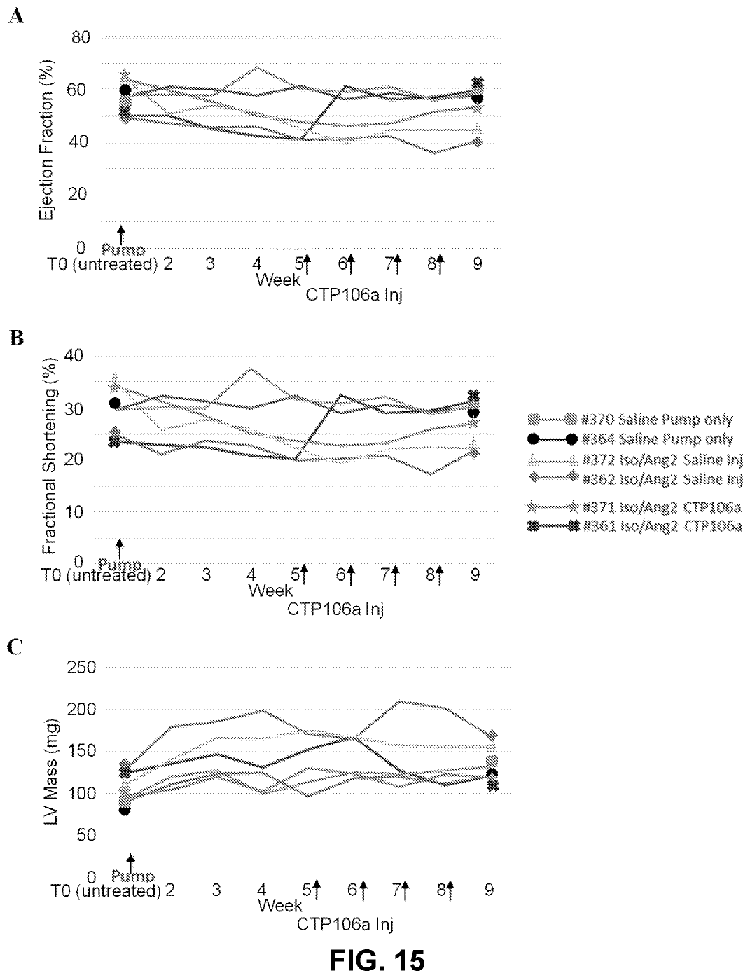

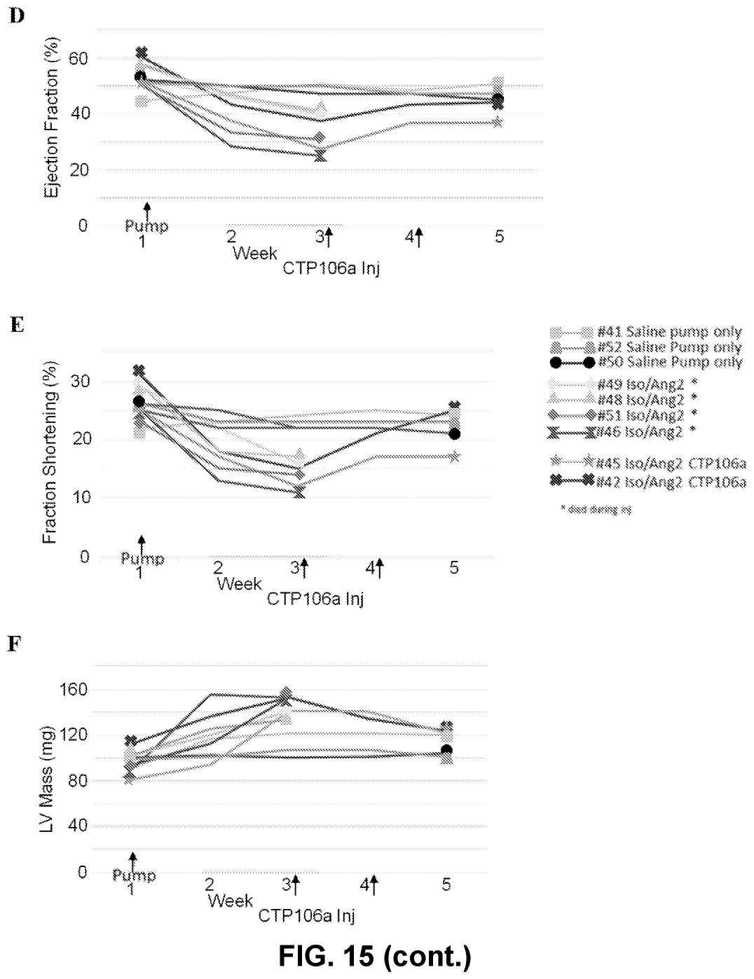

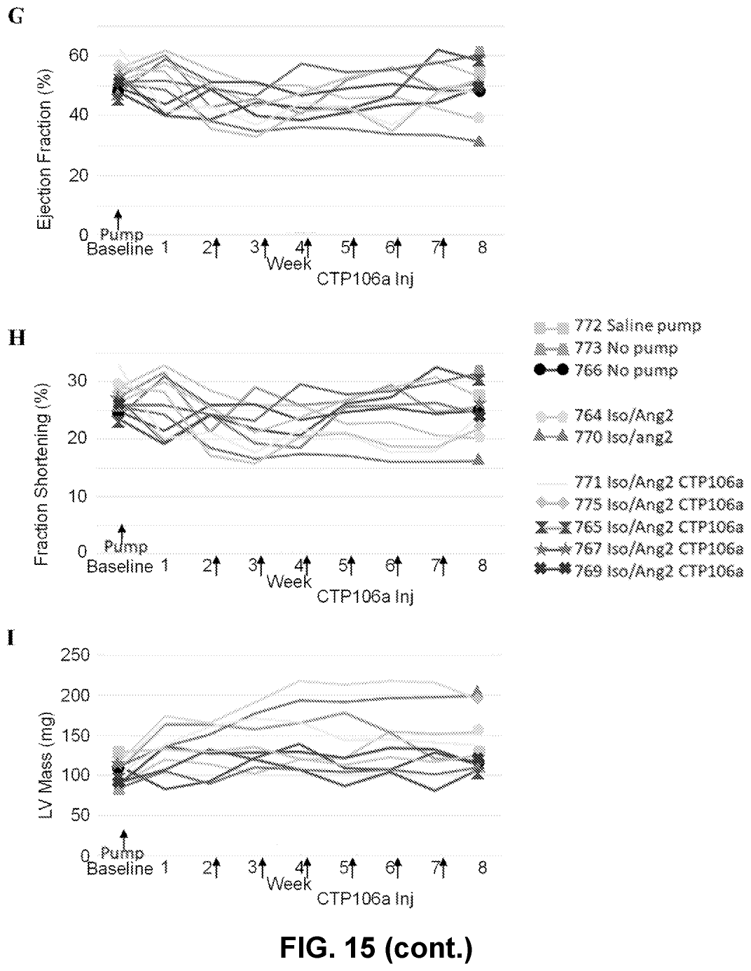

The results show that the CTP-miRNA106a conjugate reversed/rescued EF, fractional shortening (FS), and left ventricle mass (LVmass), all of which are major parameters linked to Ang2/Iso-induced heart failure (FIG. 15; Tables 5-13).

TABLE 5
EF results in mice of the first cohort.

| | | | % change | | % recovery |
|---|---|---|---|---|---|
| | | | between | | between |
| | | | Week 1 and | | Week 9 and |
| Mouse | Week 0 | Week 5 | Week 5 | Week 9 | Week 5 |
| Ang2/Iso (pump), | 66.5% | 48.5% | −27.0% | 48.5% | 0% |
| saline injection | | | | | |
| Ang2/Iso (pump), | 50.5% | 40% | −21.0% | 40% | 0% |
| saline injection | | | | | |
| Ang2/Iso (pump), | 65% | 50.5% | −22.3% | 58% | 13% |
| CTP-microRNA106a | | | | | |
| injection | | | | | |
| Ang2/Iso (pump), | 50% | 40% | −20% | 64.5% | 38% |
| CTP-microRNA106a | | | | | |
| injection | | | | | |
| Saline only (pump) | 58% | 63% | 8.6% | 63% | 0% |
| Saline only (pump) | 58% | 64% | 10% | 64% | 0% |

TABLE 6
FS results in mice of the first cohort.

| | | | % change | | % recovery |
|---|---|---|---|---|---|
| | | | between Week | | between Week |
| Mouse | Week 0 | Week 5 | 1 and Week 5 | Week 9 | 9 and Week 5 |
| Ang2/Iso (pump), | 35.5% | 24.5% | −31% | 23% | −6.1% |
| saline injection | | | | | |
| Ang2/Iso (pump), | 25.5% | 21% | −18% | 21% | 0% |
| saline injection | | | | | |
| Ang2/Iso (pump), | 35% | 24.5% | −30% | 28.5% | 14% |
| CTP-microRNA106a | | | | | |
| injection | | | | | |
| Ang2/Iso (pump), | 24% | 20% | −17% | 33.5% | 43.6% |
| CTP-microRNA106a | | | | | |
| injection | | | | | |
| Saline only (pump) | 30% | 32% | 2% | 33% | 3% |
| Saline only (pump) | 30% | 33% | 3% | 33% | 0% |

TABLE 7
LVmass results in mice of the first cohort.

| | | | % change | | % recovery |
|---|---|---|---|---|---|
| | | | between Week | | between Week |
| Mouse | Week 0 | Week 5 | 1 and Week 5 | Week 9 | 9 and Week 5 |
| Ang2/Iso (pump), | 112 mg | 165 mg | −32.1% | 160 mg | 3% |
| saline injection | | | | | |
| Ang2/Iso (pump), | 128 mg | 163 mg | −21.5% | 175 mg | 6.8% |
| saline injection | | | | | |
| Ang2/Iso (pump), | 88 mg | 125 mg | −29.6% | 110 mg | 12.5% |
| CTP-microRNA106a | | | | | |
| injection | | | | | |
| Ang2/Iso (pump), | 120 mg | 151 mg | −20.5% | 115 mg | 24% |
| CTP-microRNA106a | | | | | |
| injection | | | | | |
| Saline only (pump) | 87 mg | 108 mg | 13% | 118 mg | 8.5% |
| Saline only (pump) | 86 mg | 95 mg | 9% | 104 mg | 8.6% |

TABLE 8
EF results in mice of the second cohort (* denotes mouse died before Week 9).

| | | | % change | | % recovery |
|---|---|---|---|---|---|
| | | | between Week | | between Week |
| Mouse | Week 0 | Week 5 | 1 and Week 5 | Week 9 | 9 and Week 5 |
| Ang2/Iso (pump), | 58.5% | 41.5% | −29.0% | * | * (no recovery) |
| saline injection | | | | | |
| Ang2/Iso (pump), | 50.5% | 40% | −21.0% | * | * (no recovery) |
| saline injection | | | | | |
| Ang2/Iso (pump), | 50% | 35% | −30% | * | * (no recovery) |
| saline injection | | | | | |
| Ang2/Iso (pump), | 50% | 26% | −48% | * | * (no recovery) |
| saline injection | | | | | |
| Ang2/Iso (pump), | 51% | 27% | −47.3% | 37% | 27% |
| CTP-microRNA106a | | | | | |
| injection | | | | | |
| Ang2/Iso (pump), | 60% | 40% | −33.3% | 49.5% | 19.2% |
| CTP-microRNA106a | | | | | |
| injection | | | | | |
| Saline only (pump) | 47% | 51% | 7.8% | 52% | 2% |
| Saline only (pump) | 50% | 51% | 2% | 50% | 2% |
| Saline only (pump) | 50% | 48% | −4% | 48% | 0% |

TABLE 9
FS results in mice of the second cohort (* denotes mouse died before Week 9).

| | | | % change | | % recovery |
|---|---|---|---|---|---|
| | | | between Week | | between Week |
| Mouse | Week 0 | Week 5 | 1 and Week 5 | Week 9 | 9 and Week 5 |
| Ang2/Iso (pump), | 28.5% | 16% | −44.0% | * | * (no recovery) |
| saline injection | | | | | |
| Ang2/Iso (pump), | 28.5% | 16.5% | −42.0% | * | * (no recovery) |
| saline injection | | | | | |
| Ang2/Iso (pump), | 23% | 14.5% | −37% | * | * (no recovery) |
| saline injection | | | | | |
| Ang2/Iso (pump), | 25% | 11% | −56% | * | * (no recovery) |
| saline injection | | | | | |
| Ang2/Iso (pump), | 25% | 12.5% | −50% | 17% | 27% |
| CTP-microRNA106a | | | | | |
| injection | | | | | |
| Ang2/Iso (pump), | 31% | 15% | −51.6% | 25% | 40% |
| CTP-microRNA106a | | | | | |
| injection | | | | | |
| Saline only (pump) | 23% | 24.5% | 6.1% | 24% | −2% |
| Saline only (pump) | 26.5% | 24% | −9.4% | 24% | 0% |
| Saline only (pump) | 26% | 23.5% | −9.6% | 22.5% | −4.3% |

TABLE 10
LVmass results in mice of the second cohort (* denotes mouse died before Week 9).

| | | | | | % change | | % recovery |
|---|---|---|---|---|---|---|---|
| | | | | | between Week | | between Week |
| Mouse | Week 0 | | Week 5 | | 1 and Week 5 | Week 9 | 9 and Week 5 |
| Ang2/Iso (pump), | 99 | mg | 138 | mg | 28.3% | * | * (no recovery) |
| saline injection | | | | | | | |
| Ang2/Iso (pump), | 100 | mg | 134 | mg | 25.4% | * | * (no recovery) |
| saline injection | | | | | | | |
| Ang2/Iso (pump), | 92 | mg | 148 | mg | 37% | * | * (no recovery) |
| saline injection | | | | | | | |
| Ang2/Iso (pump), | 89 | mg | 147 | mg | 48% | * | * (no recovery) |
| saline injection | | | | | | | |
| Ang2/Iso (pump), | 80 | mg | 140 | mg | 42.8% | 118 mg | 16% |
| CTP-microRNA106a | | | | | | | |
| injection | | | | | | | |
| Ang2/Iso (pump), | 112 | mg | 154 | mg | 33.3% | 119 mg | 22.7% |
| CTP-microRNA106a | | | | | | | |
| injection | | | | | | | |
| Saline only (pump) | 100 | mg | 118 | mg | 15.3% | 119 mg | 1% |
| Saline only (pump) | 99.5 | mg | 105 | mg | 5.2% | 103 mg | 2% |
| Saline only (pump) | 99 | mg | 96 | mg | −3% | 99 mg | 0% |

TABLE 11
EF results in mice of the third cohort.

| | | | % change | | % recovery |
|---|---|---|---|---|---|
| | | | between Week | | between Week |
| Mouse | Week 0 | Week 5 | 1 and Week 5 | Week 9 | 9 and Week 5 |
| Ang2/Iso (pump), | 58% | 54% | −7% | 31% | −42.5% |
| saline injection | | | | | |
| Ang2/Iso (pump), | 50% | 39% | −22% | 38.5% | −2.5% |
| saline injection | | | | | |
| Ang2/Iso (pump), | 61% | 45% | −26.2% | 48.5% | 8% |
| CTP-microRNA106a | | | | | |
| injection | | | | | |
| Ang2/Iso (pump), | 56% | 35% | −37.5% | 52.3% | 33% |
| CTP-microRNA106a | | | | | |
| injection | | | | | |
| Ang2/Iso (pump), | 52% | 43% | −17.3% | 50.5% | 15% |
| CTP-microRNA106a | | | | | |
| injection | | | | | |
| Ang2/Iso (pump), | 52% | 48% | −8% | 50% | 4% |
| CTP-microRNA106a | | | | | |
| injection | | | | | |
| Ang2/Iso (pump), | 48% | 38% | −21% | 58.1 | 35% |
| CTP-microRNA106a | | | | | |
| injection | | | | | |
| Saline only (pump) | 51% | 50% | −2% | 52% | 3% |
| No pump | 51% | 49.5% | −2.5% | 60% | 16% |
| No pump | 47% | 51% | 7.8% | 48% | 2% |

TABLE 12
FS results in mice of the third cohort.

| | | | % change | | % recovery |
|---|---|---|---|---|---|
| | | | between Week | | between Week |
| Mouse | Week 0 | Week 5 | 1 and Week 5 | Week 9 | 9 and Week 5 |
| Ang2/Iso (pump), | 26% | 18.4% | −30% | 16.2% | −12% |
| saline injection | | | | | |
| Ang2/Iso (pump), | 29% | 28% | −3.4% | 20.2% | −28% |
| saline injection | | | | | |
| Ang2/Iso (pump), | 32% | 21% | −34% | 25% | 16% |
| CTP-microRNA106a | | | | | |
| injection | | | | | |
| Ang2/Iso (pump), | 29% | 17% | −41% | 22% | 22.7% |
| CTP-microRNA106a | | | | | |
| injection | | | | | |
| Ang2/Iso (pump), | 27% | 20.5% | −24% | 26% | 21.2% |
| CTP-microRNA106a | | | | | |
| injection | | | | | |
| Ang2/Iso (pump), | 27.5% | 24% | −19% | 24.5% | 2% |
| CTP-microRNA106a | | | | | |
| injection | | | | | |
| Ang2/Iso (pump), | 24% | 23% | −4.1% | 30% | 23.3% |
| CTP-microRNA106a | | | | | |
| injection | | | | | |
| Saline only (pump) | 28% | 27.5% | −1.7% | 27% | −2% |
| No pump | 25% | 24.8% | −1% | 30% | 16% |
| No pump | 25.5% | 26% | 2% | 24.5% | 5% |

TABLE 13
LVmass results in mice of the third cohort.

| | | | | | % change | | | % recovery |
|---|---|---|---|---|---|---|---|---|
| | | | | | between Week | | | between Week |
| Mouse | Week 0 | | Week 5 | | 1 and Week 5 | Week 9 | | 9 and Week 5 |
| Ang2/Iso (pump), | 89.4 | mg | 115.1 | mg | 22.3% | 154 | mg | 25.3% |
| saline injection | | | | | | | | |
| Ang2/Iso (pump), | 112 | mg | 150.5 | mg | 25.5% | 199.4 | mg | 24.5% |
| saline injection | | | | | | | | |
| Ang2/Iso (pump), | 103 | mg | 161 | mg | 36% | 137.5 | mg | −14.6% |
| CTP-microRNA106a | | | | | | | | |
| injection | | | | | | | | |
| Ang2/Iso (pump), | 116 | mg | 165 | mg | 29.6% | 190 | mg | 13.2% |
| CTP-microRNA106a | | | | | | | | |
| injection | | | | | | | | |
| Ang2/Iso (pump), | 108.3 | mg | 163.6 | mg | 33.8% | 118.5 | mg | −27.6% |
| CTP-microRNA106a | | | | | | | | |
| injection | | | | | | | | |
| Ang2/Iso (pump), | 89.2 | mg | 132.1 | mg | 32.5% | 113.1 | mg | −14% |
| CTP-microRNA106a | | | | | | | | |
| injection | | | | | | | | |
| Ang2/Iso (pump), | 91.7 | mg | 132.6 | mg | 31% | 107.6 | mg | −12% |
| CTP-microRNA106a | | | | | | | | |
| injection | | | | | | | | |
| Saline only (pump) | 129.1 | mg | 129.7 | mg | 0% | 125.1 | mg | −3% |
| No pump | 89.5 | mg | 89.1 | mg | −0.5% | 100.1 | mg | 10% |
| No pump | 110 | mg | 93 | mg | 15.5% | 112.3 | mg | 16.5% |

### REFERENCES

Adams B. D., et al., Targeting noncoding RNAs in disease, J. Clin. Invest., 2017. 127 (3): 761-771.
Gambardella J., et al., Role of endothelial G protein-coupled receptor kinase 2 in angioedema. Hypertension, 2020, 76 (5): 1625-1636.
Guan X., et al., miR-106a promotes cardiac hypertrophy by targeting mitofusin 2. J. Mol. Cell. Cardiol., 2016. 99:207-217.
Hohl, M., et al., HDAC4 controls histone methylation in response to elevated cardiac load, J. Clin. Invest., 2013.123 (3): 1359-1370.
Matsui M. & Corey D. R., Non-coding RNAs as drug targets, Nat. Rev. Drug Discov., 2017. 16 (3): 167-179.
Pfister E. L., et al., Safe and Efficient Silencing with a Pol II, but Not a Pol III, Promoter Expressing an Artificial miRNA Targeting Human Huntingtin, Mol. Ther. Nucleic Acids, 2017, 7:324-334.
Rupaimoole R. & Slack F. J., MicroRNA therapeutics: towards a new era for the management of cancer and other diseases, Nat. Rev. Drug Discov., 2017, 16 (3): 203-222.
Stansfield W. E., et al., Chapter 4—The Pathophysiology of Cardiac Hypertrophy and Heart Failure, Cellular and Molecular Pathobiology of Cardiovascular Disease, 2014, eds. Willis M. S., et al., Academic Press, 51-78.
van Rooij E., et al., Toward microRNA-based therapeutics for heart disease: the sense in antisense, Circ. Res., 2008, 103 (9): 919-928.

What is claimed is:

1. A method of treating cardiac hypertrophy in a subject in need thereof, the method comprising administering a pharmaceutical composition comprising an effective amount of a conjugate comprising microRNA and a cardiac targeting peptide,
wherein the microRNA is selected from miRNA106a, miRNA17, miRNA20a, and miRNA93.

2. A pharmaceutical composition comprising an effective amount of a conjugate comprising microRNA and a cardiac targeting peptide, for use in treating cardiac hypertrophy in a subject in need thereof,
wherein the microRNA is selected from miRNA106a, miRNA17, miRNA20a, and miRNA93.

3. A method of treating cardiomyocyte hypertrophy in a subject in need thereof, the method comprising administering a pharmaceutical composition comprising an effective amount of a conjugate comprising microRNA and a cardiac targeting peptide,
wherein the microRNA is selected from miRNA106a, miRNA17, miRNA20a, and miRNA93.

4. A pharmaceutical composition comprising an effective amount of a conjugate comprising microRNA and a cardiac targeting peptide, for use in treating cardiomyocyte hypertrophy in a subject in need thereof,
wherein the microRNA is selected from miRNA106a, miRNA17, miRNA20a, and miRNA93.

5. The method or pharmaceutical composition of any one of claims 1-4, wherein the cardiac hypertrophy or the cardiomyocyte hypertrophy is induced by angiotensin or phenylephrine.

6. A method of inhibiting progression of heart failure in a subject in need thereof, the method comprising administering a pharmaceutical composition comprising an effective amount of a conjugate comprising microRNA and a cardiac targeting peptide,
wherein the microRNA is selected from miRNA106a, miRNA17, miRNA20a, and miRNA93.

7. A pharmaceutical composition comprising an effective amount of a conjugate comprising microRNA and a cardiac targeting peptide, for use in inhibiting progression of heart failure in a subject in need thereof,
wherein the microRNA is selected from miRNA106a, miRNA17, miRNA20a, and miRNA93.

8. A method of reversing a reduction in cardiac function in a subject in need thereof, the method comprising administering a pharmaceutical composition comprising an effective amount of a conjugate comprising microRNA and a cardiac targeting peptide,
wherein the microRNA is selected from miRNA106a, miRNA17, miRNA20a, and miRNA93.

9. A pharmaceutical composition comprising an effective amount of a conjugate comprising microRNA and a cardiac targeting peptide, for use in reversing a reduction in cardiac function in a subject in need thereof,
wherein the microRNA is selected from miRNA106a, miRNA17, miRNA20a, and miRNA93.

10. A method of preventing a further reduction in cardiac function in a subject in need thereof, the method comprising administering a pharmaceutical composition comprising an effective amount of a conjugate comprising microRNA and a cardiac targeting peptide,
wherein the microRNA is selected from miRNA106a, miRNA17, miRNA20a, and miRNA93.

11. A pharmaceutical composition comprising an effective amount of a conjugate comprising microRNA and a cardiac targeting peptide, for use in preventing a further reduction in cardiac function in a subject in need thereof,
wherein the microRNA is selected from miRNA106a, miRNA17, miRNA20a, and miRNA93.

12. A method of inhibiting expression of Ca²⁺/calmodulin-dependent protein kinase II delta (CaMKIIδ) in cardiomyocytes, the method comprising contacting the cardiomyocytes with a conjugate comprising microRNA and a cardiac targeting peptide,
wherein the microRNA is selected from miRNA106a, miRNA17, miRNA20a, and miRNA93.

13. A conjugate comprising a microRNA and a cardiac targeting peptide, for use in inhibiting expression of Ca²⁺/calmodulin-dependent protein kinase II delta (CaMKIIδ) in cardiomyocytes,
wherein the microRNA is selected from miRNA106a, miRNA17, miRNA20a, and miRNA93.

14. A method of inhibiting expression of histone deacetylase 4 (HDAC4) in cardiomyocytes, the method comprising contacting the cardiomyocytes with a conjugate comprising a microRNA and a cardiac targeting peptide,
wherein the microRNA is selected from miRNA106a, miRNA17, miRNA20a, and miRNA93.

15. A conjugate comprising a microRNA and a cardiac targeting peptide, for use in inhibiting expression of histone deacetylase 4 (HDAC4) in cardiomyocytes,
wherein the microRNA is selected from miRNA106a, miRNA17, miRNA20a, and miRNA93.

16. The method, pharmaceutical composition, or conjugate of any one of claims 1-15, wherein the microRNA is miRNA106a.

17. The method, pharmaceutical composition, or conjugate of any one of claims 1-16, wherein the cardiac targeting peptide comprises an amino acid sequence of HLSSQYSR (SEQ ID NO: 5) or HLSSQWSR (SEQ ID NO: 18).

18. The method, pharmaceutical composition, or conjugate of any one of claims 1-17, wherein the cardiac targeting peptide has an amino acid sequence of APWHLSSQYSRT (SEQ ID NO:6).

19. The method, pharmaceutical composition, or conjugate of any one of claims 1-18, wherein the nucleic acid molecule and the CTP are linked by a covalent bond or a non-covalent bond

20. The method, pharmaceutical composition, or conjugate of any one of claims 1-18, wherein the nucleic acid molecule and the CTP are linked by a linker molecules.

21. The method, pharmaceutical composition, or conjugate of claim 20, wherein the linker molecule comprises a cleavage site
